(19) United States
(12) Patent Application Publication
(10) Pub. No.: US 2026/0053349 A1
(43) Pub. Date: Feb. 26, 2026

(54) SYSTEMS AND METHODS FOR EXAMINING TEAR FILM QUALITY USING HIGH-RESOLUTION IMAGERY

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/811,695

(22) Filed: Aug. 21, 2024

(51) Int. Cl. A61B 3/10; A61B 3/00; A61B 3/14; G16H 50/70

(52) U.S. Cl. CPC A61B 3/101; A61B 3/005; A61B 3/14; G16H 50/70

(57) ABSTRACT

A patient's tear film quality can be evaluated via a virtual reality (VR) system, which can include a VR headset in electronic communication with a computing device. The computing device can cause visual stimuli to be displayed on the screens of the VR headset. Using varying combinations of sensors, cameras, probes, and microphones, the VR headset collects tear film samples as well as data about the patient as she reacts and responds to the phenomenon. Optionally, the computing device can alter the visual stimuli and analyze the patient's reactions to conduct an ocular evaluation.

### BACKGROUND

### Field of the Inventions

The present application relates to methods of assessing various ocular conditions through extended reality systems. More specifically, methods and systems are applied to conduct visual tasks and exams in extended reality environments to evaluate patients for ocular conditions and diseases, such as eye misalignment and visual processing disorders.

### Description of the Related Art

As virtual reality (VR) technology has become increasingly sophisticated, new highly immersive experiences have been made possible through improvements in head and motion tracking systems. Eye-tracking technology allows systems to detect and respond to where the user is looking. This capability enhances user interaction and makes virtual environments more responsive and engaging. Eye tracking is being integrated into a variety of VR applications, from gaming and training simulations to medical diagnostics and research, as it offers a more intuitive way for users to interact with digital content.

### SUMMARY

The systems, methods and devices of this disclosure each have several innovative aspects, no single one of which is solely responsible for the desirable attributes disclosed herein.

Despite the advancements in VR technology, and in particular, eye-tracking technology, in accordance with some embodiments disclosed herein is the realization that VR technology can provide unique eyecare solutions through monitoring and tracking one or more of the user's eyes and providing a diagnostic, treatment protocol, and/or treatment system. Indeed, in accordance with some embodiments disclosed herein is the realization that VR technology can be used to address challenges associated with diagnosing a variety of eye disorders and ocular conditions, such as detecting misalignment, macular degeneration, tear film characteristics, floater characteristics, eye tracking issues, motion sensitivity, and other eye movement disorders and the treatment of such.

In some embodiments, a virtual reality (VR) guided examination can be conducted for the early detection of macular degeneration using detailed visual patterns. The method can be implemented on a VR system that comprises a high-resolution VR headset equipped with advanced eye-tracking sensors and software algorithms tailored for detailed visual pattern recognition. Users will wear the VR headset, which projects a series of intricate visual stimuli designed to test various aspects of visual acuity and retinal function. The eye-tracking sensors continuously monitor the patient's gaze, pupil response, and micro-movements to detect abnormalities indicative of macular degeneration.

Optionally, a VR software can employ a series of tests, including grid patterns, dot arrangements, and color differentiation tasks, which are displayed in varying levels of contrast and brightness on the screens of the VR headset. The VR software's algorithms analyze the patient's responses to these visual patterns in real-time, comparing the responses to a database of known responses from healthy individuals and individuals with early-stage macular degeneration. This comparative analysis allows the system to identify subtle deviations and patterns that may indicate the onset of the disease. The results are compiled into a detailed report that highlights any potential early signs of macular degeneration, providing valuable information for further medical evaluation.

To construct the VR macular degeneration detection system, begin with a commercially available high-resolution VR headset, such as the Oculus Rift or HTC Vive, and integrate custom-designed eye-tracking hardware capable of capturing detailed ocular movements with high precision. The eye-tracking component should include infrared sensors and cameras to monitor the pupil's response to various visual stimuli accurately. The software development aspect involves creating a library of visual tests, each designed to assess different parameters of visual function associated with macular health. These tests include but are not limited to Amsler grid variations, color discrimination patterns, and moving dot arrays.

Once the hardware and software components are integrated, in some embodiments, the system can be calibrated for accuracy using a control group of individuals with known visual health statuses. The patient can then wear the VR headset and undergo a series of visual tests within a controlled environment. The software records and analyzes the data, providing immediate feedback on the patient's visual performance. The system is designed to be user-friendly, requiring minimal technical expertise to operate, making it suitable for use in both clinical settings and remote telemedicine applications. This approach allows for early detection of macular degeneration, enabling timely intervention and potentially preserving vision in individuals at risk.

In some embodiments, a VR method for comprehensively examining tear film quality can be performed by analyzing high-resolution imagery. This method can be implemented on a sophisticated VR headset integrated with high-definition cameras and specialized sensors capable of capturing minute details of the eye's tear film. The VR headset projects immersive visual stimuli designed to induce natural blinking and tear film distribution to the patient while the embedded cameras take high-resolution images of the tear film dynamics, including tear breakup time, lipid layer thickness, and overall tear stability.

In some embodiments, the VR headset can be equipped with miniaturized, high-resolution cameras strategically positioned to capture detailed images of the tear film from multiple angles. The cameras are coupled with infrared illuminators to enhance the visibility of the tear film without causing discomfort to the patient. These images are analyzed in real-time by advanced image processing algorithms, providing detailed assessments of tear film quality and identifying any abnormalities. The captured images are processed using machine learning algorithms trained on a vast dataset of tear film characteristics, enabling accurate and detailed analysis. The results are presented in a comprehensive report, highlighting potential issues such as dry eye syndrome or other tear film-related disorders.

To create the VR tear film examination system, begin with a high-quality VR headset, such as the Oculus Quest 2, and integrate custom high-resolution cameras capable of capturing detailed imagery of the eye's surface. These cameras should have a resolution of at least 4K and be equipped with infrared illuminators to ensure clear imaging under various lighting conditions. The software is developed to include a library of visual stimuli that promote natural eye movements and blinking, essential for accurate tear film assessment. The system's machine learning algorithms are trained on a diverse dataset, encompassing a wide range of tear film conditions to ensure robust and precise analysis.

Once assembled, the VR system undergoes rigorous calibration and testing using subjects with known tear film conditions to ensure accuracy and reliability. Patients can operate the system by wearing the VR headset and following a guided series of visual tasks. The high-resolution cameras capture images of the tear film before, during, and after blinking, while the software processes these images to evaluate tear film stability, thickness, and distribution. The results are immediately available, providing a detailed assessment of tear film quality. This platform offers a non-invasive, highly accurate method for diagnosing and monitoring tear film disorders, offering substantial benefits for both clinical and telemedicine applications.

In some embodiments, a VR method for diagnosing and monitoring retinal disorders can be performed through focused vision tests. This method can be implemented on a VR system that comprises a high-fidelity VR headset equipped with precision eye-tracking technology and a suite of specialized vision tests targeting various aspects of retinal health. The headset generates immersive environments where patients undergo a series of visual assessments, such as central vision acuity tests, peripheral vision tests, color vision tests, and contrast sensitivity tests. The eye-tracking sensors meticulously monitor the patient's eye movements, fixation stability, and response times, providing comprehensive data on retinal function.

Optionally, a VR software causes a range of diagnostic tests, including dynamic visual stimuli, to be shown on screens of the VR headset to evaluate the patient's retinal health accurately. These tests are designed to detect early signs of retinal disorders such as macular degeneration, diabetic retinopathy, and retinal detachment. The system's algorithms analyze the collected data in real-time, comparing it against a database of normative values to identify deviations indicative of retinal pathology. The results are compiled into a detailed report, highlighting areas of concern and recommending further medical evaluation if necessary. This approach allows for early detection and continuous monitoring of retinal disorders, facilitating timely intervention and better patient outcomes.

In some embodiments, a VR system for diagnosing and monitoring retinal disorders can be designed to include a state-of-the-art VR headset (e.g., the Oculus Quest 2) that can be enhanced with high-precision eye-tracking technology. The eye-tracking component should include infrared sensors and cameras capable of capturing detailed ocular movements with sub-millimeter accuracy. The software development involves creating a comprehensive library of vision tests, each tailored to assess different aspects of retinal function. These tests include static and dynamic stimuli, designed to challenge the patient's visual system and elicit responses that provide insights into retinal health.

Once the hardware and software components are integrated, the system undergoes calibration using a control group of individuals with known retinal health statuses to ensure accuracy and reliability. Patients can then wear the VR headset and follow a guided series of vision tests within an immersive virtual environment.

Eye-tracking sensors can capture detailed data on eye movements, fixation stability, and visual response times, which the software analyzes to detect abnormalities. The system is designed for ease of use, requiring minimal training, making it suitable for both clinical and remote telemedicine applications. This method is a non-invasive, highly accurate method for diagnosing and monitoring retinal disorders, providing substantial benefits for early detection and ongoing management of retinal health.

In some embodiments, a VR method for assessing rapid eye movement (REM) sleep can be performed to offer insights into overall eye health and identifies potential sleep-related disorders. This method can be implemented via a VR headset integrated with high-precision eye-tracking technology and specialized software algorithms designed to monitor and analyze REM patterns during sleep. The system is capable of non-invasively recording eye movements in real-time, providing a detailed analysis of REM phases, frequency, and amplitude, which are critical indicators of both neurological function and sleep quality. The VR method involves users wearing a VR headset equipped with infrared eye-tracking sensors while sleeping. The eye-tracking technology continuously captures eye movement data, which is then processed by sophisticated software algorithms to distinguish between different sleep stages and identify REM periods. The system generates a comprehensive report that highlights any irregularities in REM patterns, such as excessive or diminished REM activity, which can be indicative of sleep disorders like narcolepsy, sleep apnea, and REM sleep behavior disorder. Additionally, the analysis provides valuable information on overall eye health, as abnormalities in REM can correlate with certain ocular conditions.

Optionally, the VR-based REM assessment system can be constructed using a high-resolution VR headset (e.g., the Oculus Quest 2), with high-precision infrared eye-tracking sensors integrated therewith. These sensors should be capable of capturing detailed ocular movements with high accuracy and minimal latency. The software component requires the development of algorithms capable of distinguishing REM sleep from other sleep stages based on eye movement patterns. The system also includes a user-friendly interface for setting up and initiating sleep monitoring sessions. Once the hardware and software components are integrated, the system is calibrated using a control group to establish baseline REM patterns and validate the accuracy of the sleep stage classification algorithms. Patients can then use the system by wearing the VR headset before going to sleep. The eye-tracking sensors monitor and record eye movements throughout the night, with the data being processed in real-time to identify REM periods and analyze their characteristics. Upon waking, patients receive a detailed report that includes an analysis of their REM sleep patterns, highlighting any abnormalities and providing insights into potential sleep-related disorders and overall eye health. This approach offers a non-invasive, accurate, and comprehensive method for assessing REM sleep, advancing patients'understanding of their sleep disorders and/or ocular health.

In some embodiments, a VR-based guided task or procedure can be implemented to identify and categorize ocular floaters by simulating various lighting conditions. This task can be carried out via a high-resolution VR headset equipped with advanced eye-tracking sensors and a sophisticated software suite that generates dynamic lighting environments. Patients wear the VR headset and are exposed to different simulated lighting conditions, such as bright sunlight, dim indoor light, and varying contrast scenarios. The eye-tracking sensors monitor the patient's eye movements and responses to these conditions, while the software analyzes the visual disturbances reported by the patient, specifically focusing on floaters. The VR system incorporates a series of diagnostic tests that prompt patients to describe the size, shape, location, and movement of floaters they perceive under different lighting conditions. The data collected is processed using advanced algorithms to categorize floaters based on their characteristics and behavior under varying illuminations. This approach allows for a detailed and nuanced understanding of floaters, providing valuable information for ophthalmologists to diagnose underlying conditions such as vitreous detachment or retinal tears. The results are compiled into a comprehensive report, detailing the types and severity of floaters, and offering recommendations for further medical evaluation if necessary.

To construct the VR-based floater identification and categorization system, start with a high-fidelity VR headset, such as the Oculus Quest 2, integrated with high-precision eye-tracking technology. The eye-tracking sensors should include infrared cameras capable of capturing detailed eye movements with high accuracy. The software component involves developing a library of lighting simulations and diagnostic tests that challenge the patient's visual system under different lighting conditions. These tests include scenarios with varying brightness, contrast, and color temperatures to elicit the perception of floaters. Once the hardware and software components are integrated, the system is calibrated using a control group of individuals with known floater conditions to establish baseline responses and validate the accuracy of the categorization algorithms. Patients can then operate the system by wearing the VR headset and following a series of guided visual tasks. The eye-tracking sensors monitor their eye movements and responses to different lighting conditions, while the software records their descriptions of perceived floaters. The data is analyzed in real-time to identify and categorize floaters, providing immediate feedback in the form of a detailed report. This report includes a classification of floaters, potential underlying causes, and suggestions for further medical consultation. This approach offers a non-invasive, highly accurate method for diagnosing and understanding ocular floaters, offering substantial benefits for patient care.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is an example data processing environment having one or more servers communicatively coupled to one or more computer devices, in accordance with some embodiments.

FIG. 2 is an environment in which a computer device is applied to facilitate visual assessment or eyewear fitting, in accordance with some embodiments.

FIG. 3 is a block diagram of a computer system configured to implement vision assessment or eyewear fitting, in accordance with some embodiments.

FIG. 4 is a block diagram of a machine learning system for training and applying machine learning models, in accordance with some embodiments.

FIG. 5A is a structural diagram of an example neural network applied to process input data in a machine learning model, in accordance with some embodiments.

FIG. 5B is an example node in the neural network, in accordance with some embodiments.

FIG. 6A is an example “tumbling E” chart applied in a visual acuity test, in accordance with some embodiments.

FIGS. 6B, 6C, 6D, and 6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIG. 11A illustrates the conventional method of evaluating a patient for macular degeneration, in accordance with some embodiments disclosed herein.

FIG. 11B illustrates a VR headset for evaluating a patient for macular degeneration using a VR method, in accordance with some embodiments disclosed herein.

FIGS. 11C-11D each illustrate progression of visual tests that can be administered as part of the dynamic macular degeneration evaluation, in accordance with some embodiments disclosed herein.

FIGS. 11E-11F illustrate a VR headset with different images being shown on each screen so that a patient can individually adjust the contrast of each image until the images have the same contrast, in accordance with some embodiments described herein.

FIG. 12A illustrates a VR system for examining tear film quality using high-resolution imagery, in accordance with some embodiments disclosed herein.

FIG. 12B illustrates the position of the cameras on the VR headset relative to the patient's eyes, in accordance with some embodiments described herein.

FIG. 13 illustrates a VR environment in which the patient can create VR floaters, in accordance with some embodiments described herein.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

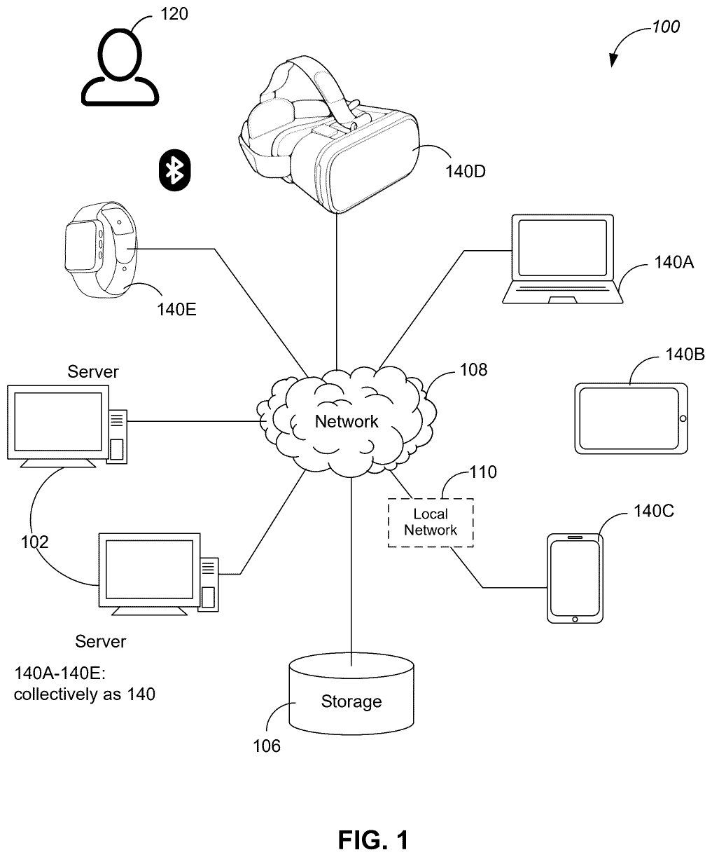

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D (also called a head-mounted display (HMD) device 140D) configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real-time data communication with the computer devices 140 that can be remote from each other or from the one or more servers 102. Further, in some embodiments, the one or more servers 102 can implement data processing tasks that are not completed locally by the computer devices 140. For example, the computer devices 140 can include a game console (e.g., the headset device 140D) that executes an interactive online gaming application (e.g., for visual assessment or eyewear fitting). The game console receives a user instruction and sends it to a server 102 with user data. The server 102 generates a stream of video data based on the user instruction and user data and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound includes user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern. For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

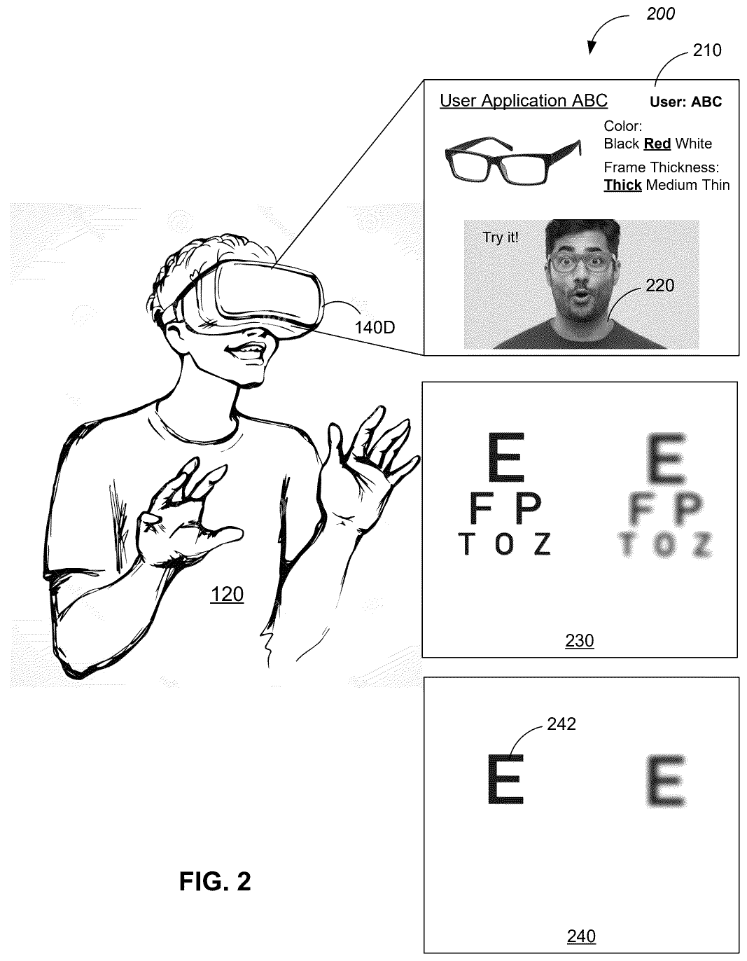

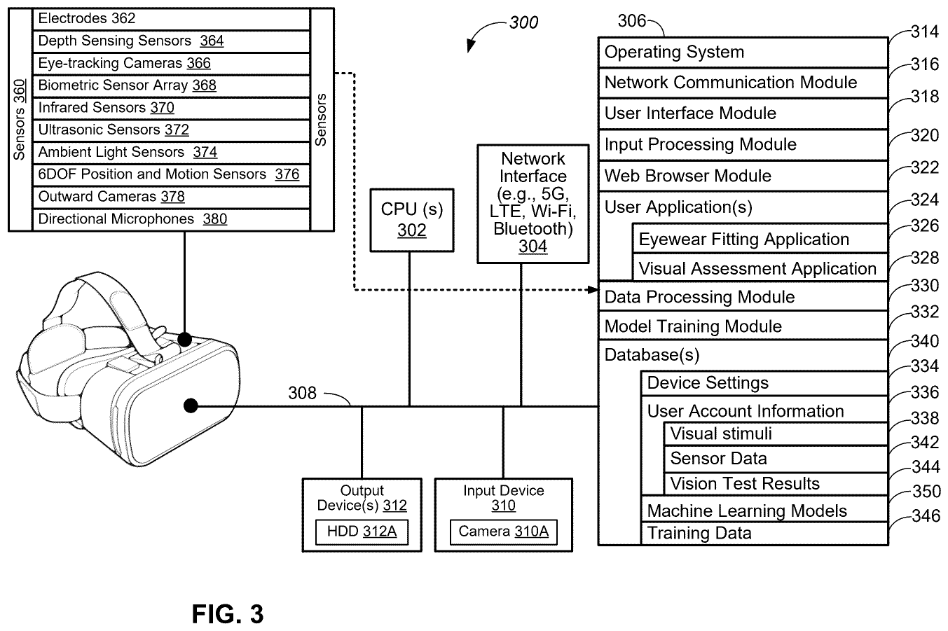

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive, from the first user interface 210, a user feedback message indicating an issue, requesting further improvement, or confirming a fit. In some embodiments, a second user interface 230 can be displayed on a computer device 140 associated with the user 120. The second user interface 230 may include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 may include one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer device 140 of the computer system 300 may use a microphone for voice recognition or an eye tracking camera 366 for tracking eyeball movement. In some implementations, the computer device 140 may include one or more optical cameras (e.g., an RGB camera), scanners, or photo sensor units for capturing images. The computer system 300 may also include one or more output devices 312 that enable presentation of user interfaces 210 and media content. The one or more output devices 312 may include one or more speakers and/or one or more visual displays.

The computer system 300 may include one or more sensors 360, which further may include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more directional microphones 380. It is noted that the one or more sensors 360 can also be included in the input device 310 and used to collect data to the computer system 300.

Memory 306 may include high-speed random-access memory, such as DRAM, SRAM, DDR RAM, or other random-access solid state memory devices; and, optionally, may include non-volatile memory, such as one or more magnetic disk storage devices, one or more optical disk storage devices, one or more flash memory devices, or one or more other non-volatile solid state storage devices. Memory 306, optionally, may include one or more storage devices remotely located from one or more processing units 302. Memory 306, or alternatively the non-volatile memory within memory 306, may include a non-transitory computer readable storage medium. In some implementations, memory 306, or the non-transitory computer readable storage medium of memory 306, may store the following programs, modules, and data structures, or a subset or superset thereof:

Operating system 314 including procedures for handling various basic system services and for performing hardware dependent tasks;

Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;

User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);

Input processing module 320 for detecting one or more user inputs or interactions from one of the one or more input devices 310 and interpreting the detected input or interaction;

Web browser module 322 for navigating, requesting (e.g., via HTTP), and displaying websites and web pages thereof may include a web interface for logging into a user account associated with a computer device 140 or another electronic device, controlling the computer device if associated with the user account, and editing and reviewing settings and data that are associated with the user account;

One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);

Data processing module 330 for processing data associated with the user applications 324, e.g., using machine learning models 350;

Model training Module 332 for obtaining training data 346 and training machine learning models 350; and

One or more databases 340 for storing at least data including one or more of:

Device settings 334 including common device settings (e.g., service tier, device model, storage capacity, processing capabilities, communication capabilities, etc.) of the computer system 300;

User account information 336 for the one or more user applications 324, e.g., user names, security questions, account history data, user preferences, and predefined account settings, where in some embodiments, the user account information 336 may include facial measurements and one or more virtual fitting parameters associated with associated with a user account of an eye fitting application 326, and in some embodiments, the user account information 336 may include visual stimuli 338, sensor data 342, and vision test results 344 associated with a user account of a visual assessment application 328; and

Machine learning models 350 including parameters (e.g., weights, biases) used to implement vision test or select eyewear for eyewear users.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in some embodiments. In some embodiments, memory 306, optionally, stores a subset of the modules and data structures identified above. Furthermore, memory 306, optionally, stores additional modules and data structures not described above.

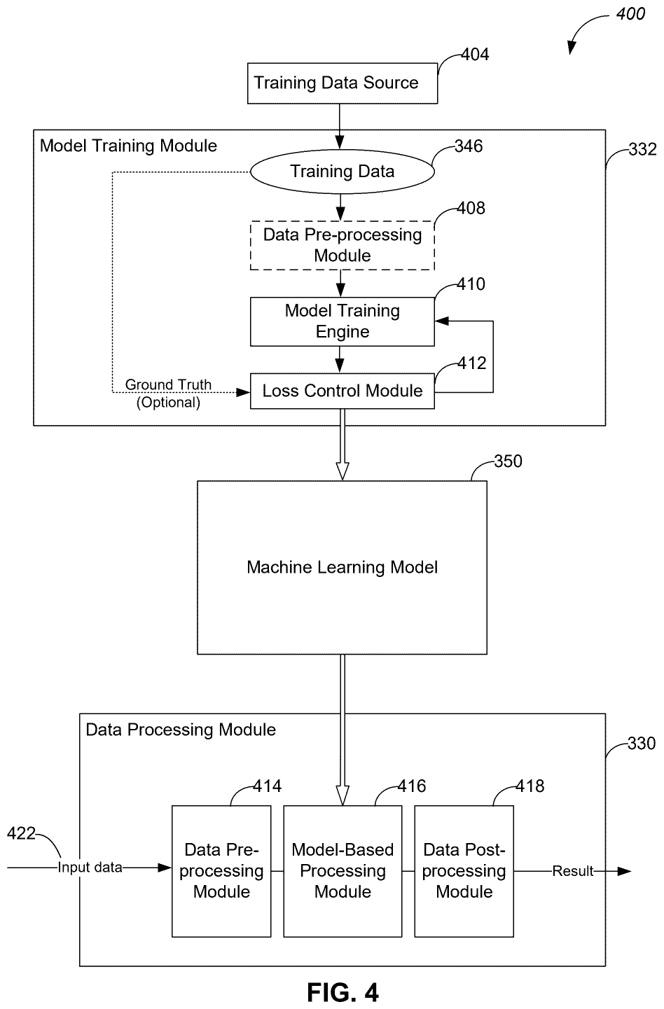

FIG. 4 is a block diagram of a machine learning system 400 for training and applying machine learning models 350 (e.g., for glass making), in accordance with some embodiments. The machine learning system 400 may include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 may include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real-time input data 422 detected by the computer device 140. In some embodiments, the training data 346 provided by the training data source 404 may include a standard dataset widely used to train machine learning models 350. The input data 422 further may include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 may include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criterion (e.g., a comparison result of the loss function is minimized or reduced below a loss threshold). The machine learning models 350 may thereby be trained and provided to the data processing module 330 of a computer device 140 to process real-time input data 422 from the computer device 140.

In some embodiments, the model training module 402 may further include a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and include a desired output for each training data item (also called the ground truth, in some embodiments). In some embodiments, the desirable output may be labelled manually by people or automatically by the model training model 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 may include a data pre-processing module 414, a model-based processing module 416, and a data post-processing module 418. The data pre-processing modules 414 may pre-process input data 422 based on the type of the input data 422. In some embodiments, functions of the data pre-processing modules 414 are consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

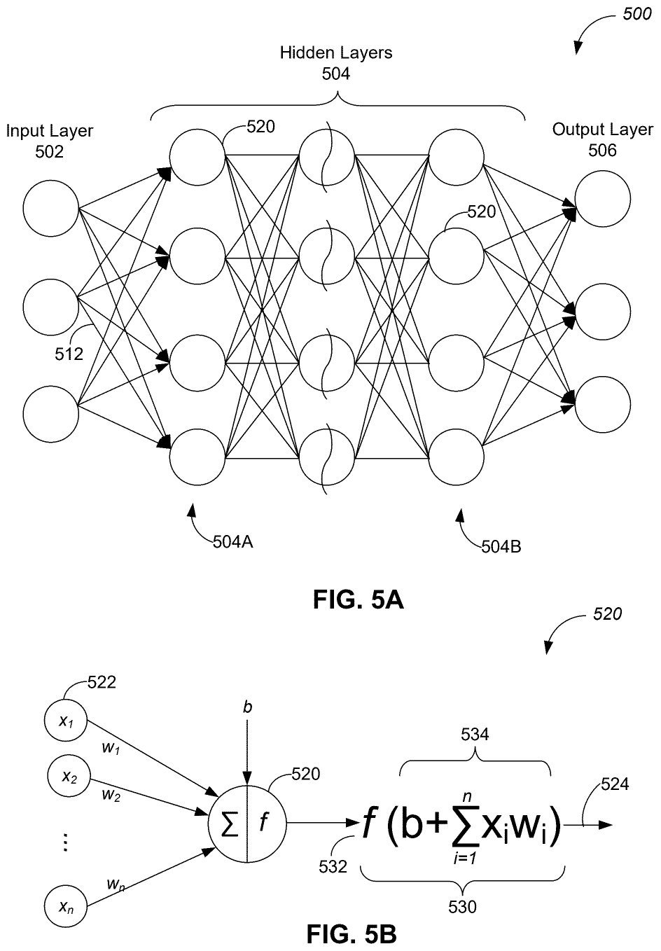

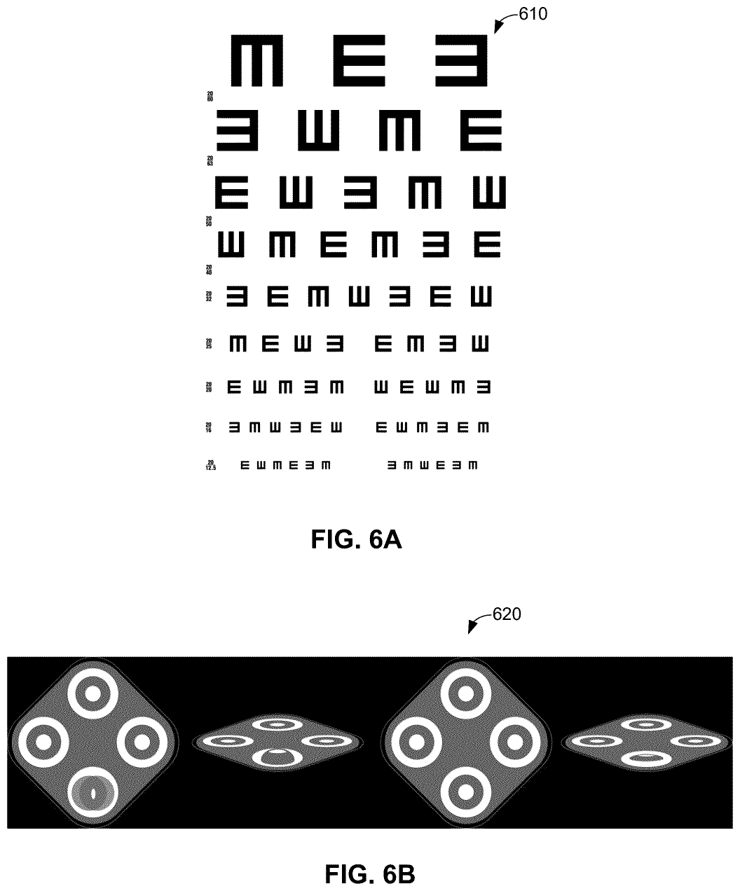

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 may include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers may include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a “fully connected” layer because each node in the layer is connected to every node in its immediately following layer. In some embodiments, a hidden layer 504 may include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN include convolutional layers. Each node in a convolutional layer may receive inputs from a receptive area associated with a previous layer (e.g., nine nodes). Each convolution layer may use a kernel to combine pixels in a respective area to generate outputs. For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may be abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights wᵢ for each layer of the neural network 500 using training data 346 that is provided in the input layer 502. The training process typically may include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training may include a network bias parameter b for each layer.

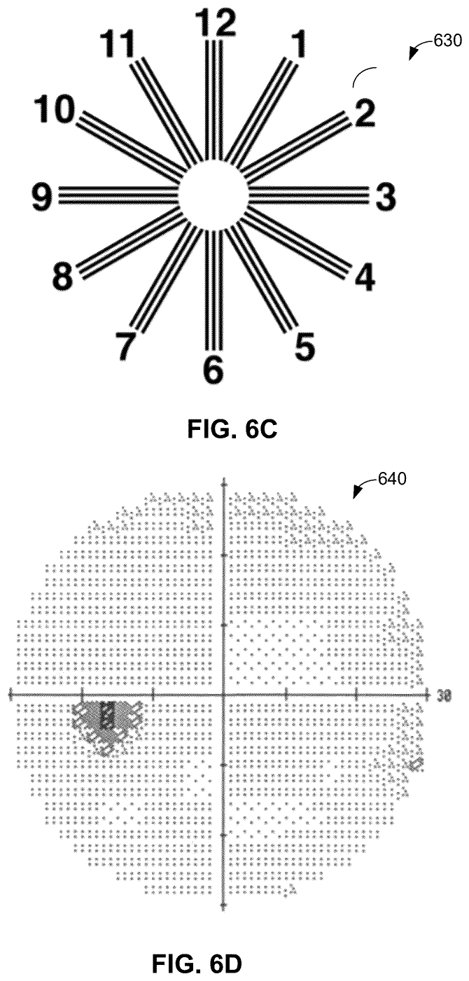

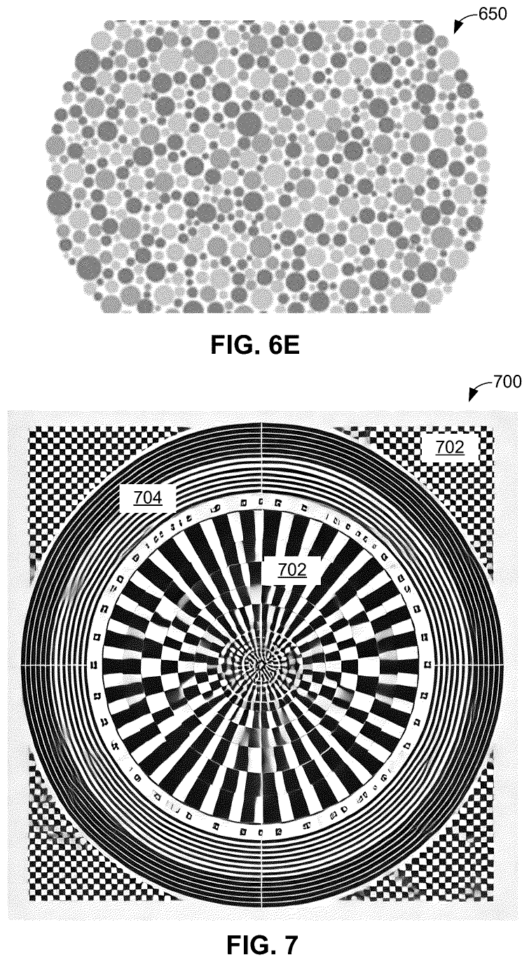

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example “tumbling E” chart 610 applied in a visual acuity test, in accordance with some embodiments. FIGS. 6B, 6C, 6D, and 6E are example patterns 620, 630, 640, and 650 applied in an astigmatism test, a stereopsis test, a visual field test, and a color blindness test, in accordance with some embodiments.

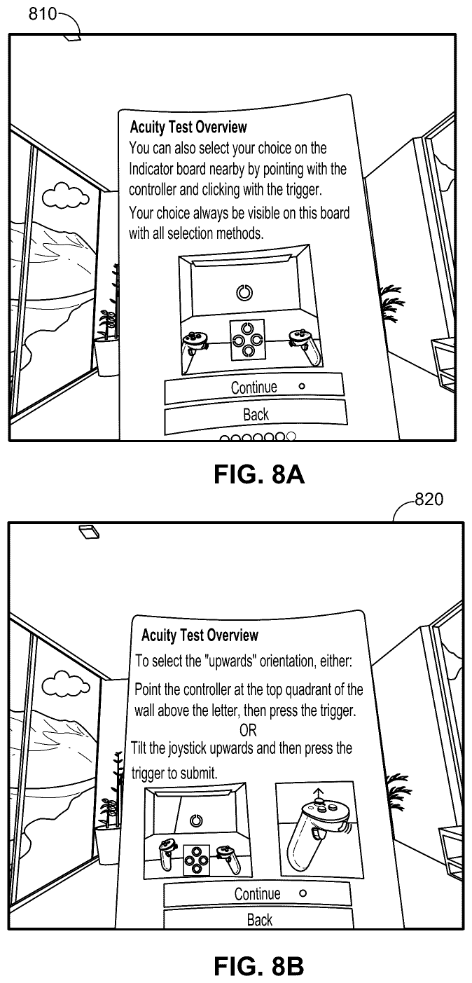

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

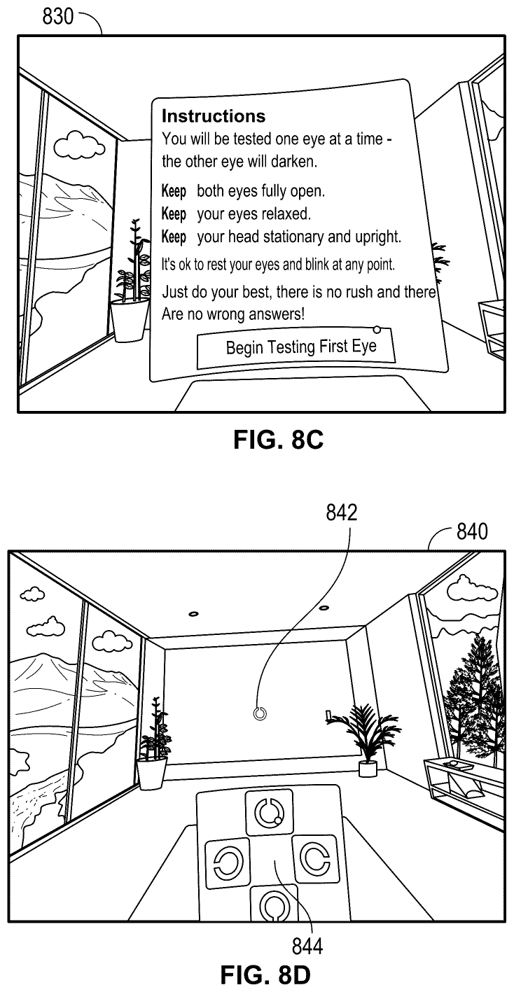

FIGS. 8A-8D include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using the controller to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

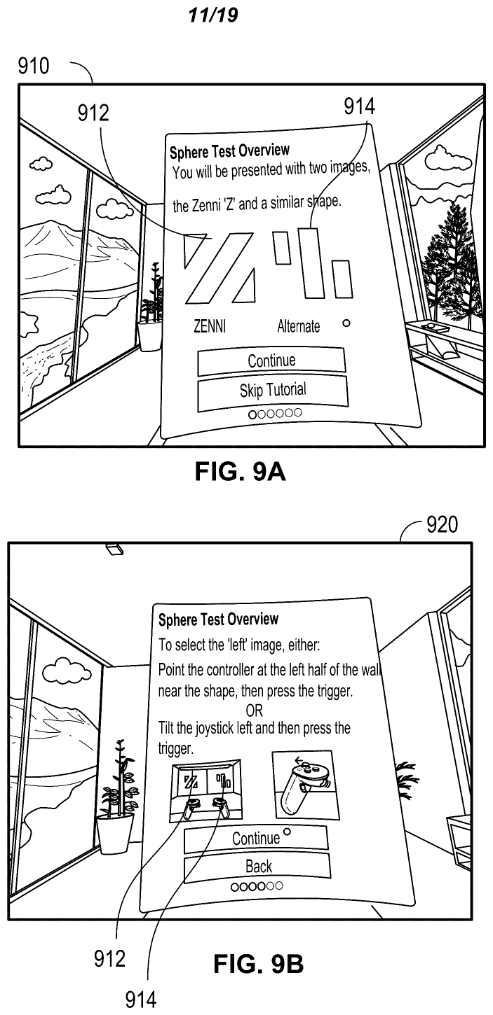

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using the controller to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel 932 may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

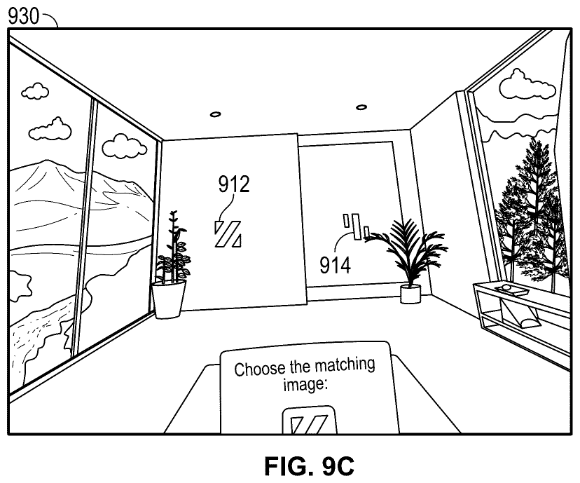

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 may include a message, e.g., “You will be presented with a clock diagram of converging numbered lines. ” The user interface 1020 may display an information page explaining what is selected on the clock diagram of converging numbered lines 1012 displayed in the virtual environment. For example, the user interface 1010 may include a message, e.g., “Your task is to identify if any of these sets of lines appear clearer, crisper, or darker than other.” The user interface 1030 may display an information page including two optional ways of using the controller to select lines on the clock diagram of converging numbered lines 1012. For example, the user interface 1010 may include a message, e.g., “Make a selection by either pointing the controller at the lines on the clock, then pressing the trigger” and “Rotating the joystick to move the indicator arrows around the clock.” The user interface 1040 may display an information page illustrating an embodiment having equally clear lines on the clock diagram of converging numbered lines 1012. For example, the user interface 1010 may include a message, e.g., “If two sets of neighboring lines seem to both stand out as equally clear, you can move the indicator arrows to a halfway point between those lines.”

Referring to FIG. 10E, the user interface 1050 may display an information page including an instruction using the controller to submit a selection. For example, the user interface 1010 may include a message, e.g., “After selecting a set of lines, submit your choice with the ‘Done’ button below by pointing to the controller at the button and pressing the trigger. ” Further, referring to FIG. 10F, the user interface 1060 may display an information page including an instruction using the controller to indicate that no difference is observed on the clock diagram of converging numbered lines 1012. For example, the user interface 1010 may include a message, e.g., “It's important to understand that not everybody will see a difference between the lines” and “In this case, simply select ‘No Difference’ below, by positioning the controller at the button and pressing the trigger.”

### Detecting Early Signs of Macular Degeneration Using Detailed Visual Patterns

Macular degeneration is an eye disease that affects a person's macula, which is the central part of the retina. As a result, a person with macular degeneration might have reduced central vision, which makes it difficult to see things that are directly in front of her. Macular degeneration can start even before the person experiences vision loss that can be assessed by typical diagnostic exams. However, early detection of macular degeneration is critical because treating macular degeneration in its early stages can meaningfully slow the progression of the macular degeneration, which decreases the severity of the associated vision loss.

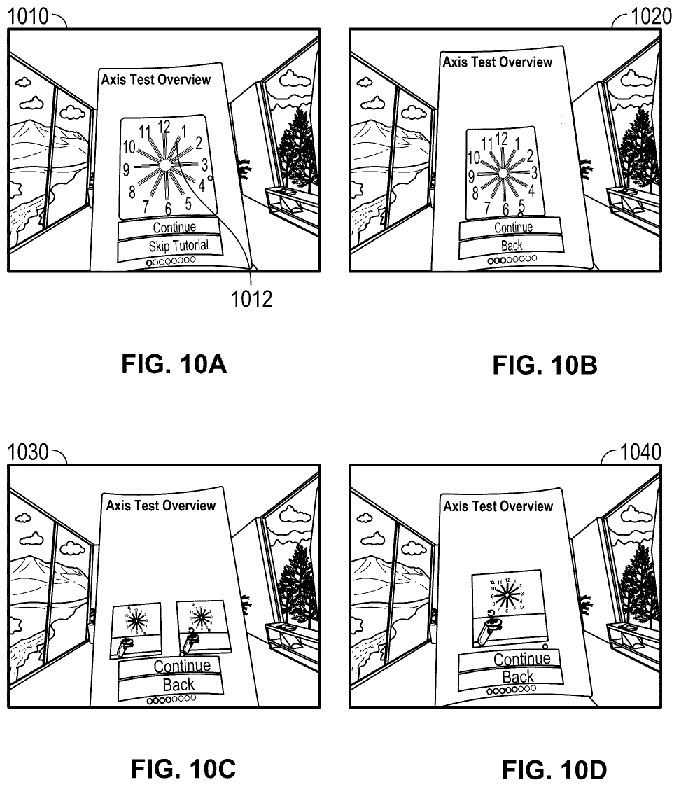

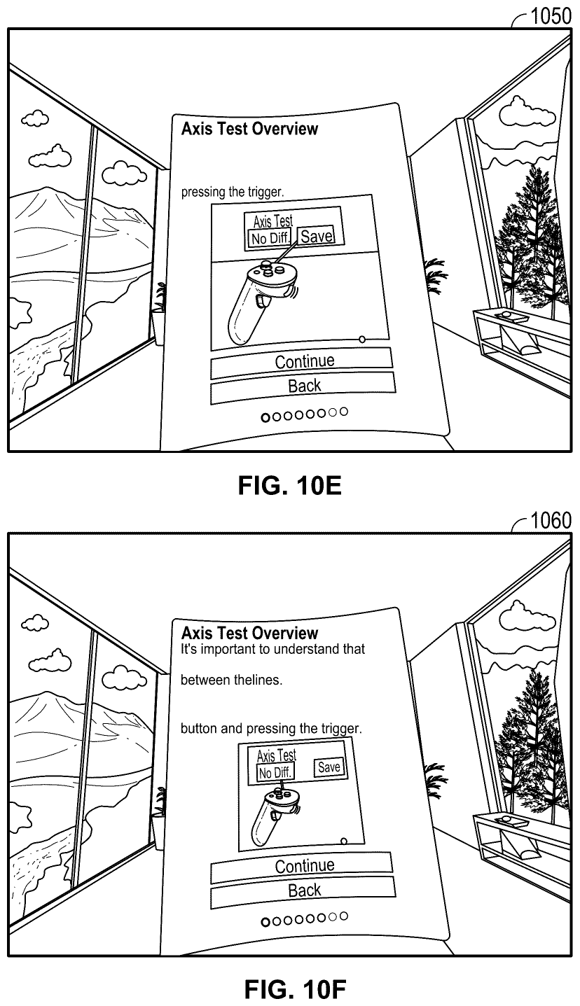

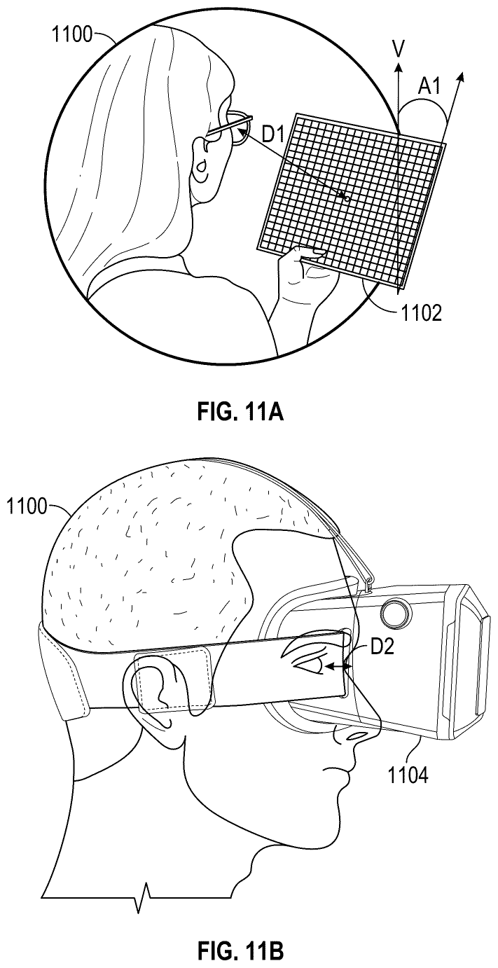

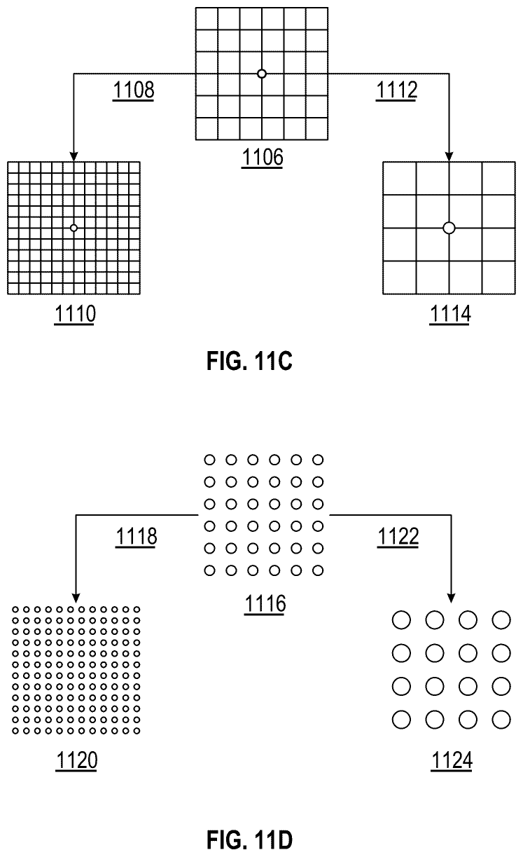

FIG. 11A illustrates the conventional method of evaluating a patient for macular degeneration, in accordance with some embodiments disclosed herein. Macular degeneration is most commonly evaluated using an Amsler grid, which is a grid of straight lines with a dot in the center. A physician typically hands a patient 1100 a piece of paper with an Amsler grid printed 1102 on it. The patient will hold the paper at arm's length with one hand, cover one eye with the other hand, look at the dot in the center of the Amsler grid 1102, and tell the physician which areas of the Amsler grid 1102 are not visible and which areas of the Amsler grid 1102 look blurry, wavy, etc. A patient's indication that the grid has a lot of missing, blurry, or wavy areas, is a significant sign that the patient has macular degeneration.

However, the typical macular degeneration evaluation is not always accurate due to inconsistencies related to the administration of the evaluation. For example, the distance D1 between the eyes of the patient 1100 and the Amsler grid 1102 can vary from patient to patient because some patients might hold the Amsler grid 1102 with their arms fully extended while others might have their arms bent, as shown in FIG. 11A. The distance D1 can also vary due to different patients having different arm lengths or due to the patient 1100 moving (i.e., leaning forwards or backwards, bending or straightening her arm, etc.) during the evaluation. Moreover, the angle A at which the patient 1100 holds the Amsler grid 1102 relative to a vertical axis V can also vary because it is difficult for physicians to make all patients hold the Amsler grids at the same angle. The tilt of the head of the patient 1100 can also vary between patients as well as throughout the evaluation of an individual patient, which adds another element of inconsistency to the exam. Finally, the means by which the patient 1100 indicates which portions of the Amsler grid 1102 are missing, blurry, or wavy can be very subjective if the patient 1100 is verbally describing which portions of the Amsler grid 1102 are missing, blurry, or wavy. There are even inconsistencies if the patient 1100 circles or highlights the portions of the Amsler grid 1102 that are missing, blurry, or wavy because circling or highlighting the Amsler grid 1102 requires the patient 1100 to remove her second hand from her eye and put the Amsler grid 1102 on a writing surface to mark the Amsler grid 1102. This readjustment of the perspective and the Amsler grid 1102 reduces the accuracy of the patient's 1100 markings.

FIG. 11B illustrates a VR headset for evaluating a patient for macular degeneration using a VR method, in accordance with some embodiments disclosed herein. In a preferred embodiment of the method disclosed, the patient 1100 dons a headset such as VR headset 1104. The VR headset 1104 has screens for displaying a VR environment to the patient 1100 as well as one or more sensors that collect data about and input from the patient 1100 as the patient 1100 perceives and reacts to the VR environment. The sensors may be constructed and operable in accordance with any variety of conventional technologies. In some embodiments, the VR headset includes, in addition to the sensors, cameras and microphones that further collect data about and input from the patient. The sensors and cameras can be used to monitor the data such as pupil dilation, blink rates, and response as well as inputs such as eye movements as the patient 1100 reacts and responds to the VR environment. Optionally, the sensors and cameras can be used to collect biodata such as heart rate, scarring on or around the eyes, skin conductance, etc. Meanwhile the microphones can be used to receive verbal responses to questions asked during the evaluation.

The VR headset 1104 is configured to be in electronic communication with a computing device, which, in turn, is configured to process the data and input received by the sensors, cameras, and microphones. The computing device also controls the environment displayed on the screens of the VR headset 1104, and the environment displayed can be influenced by the data and the input. In some embodiments, the computing device includes a user interface at which a physician can view the results of the macular degeneration evaluation as the computing device processes the data and input (i.e., in real-time). This allows the physician to monitor and adjust the evaluation, diagnose the patient with macular degeneration, or otherwise provide insights as to the patient's visual health. Optionally, the patient can use the user interface to view her results in a homecare setting.

In some embodiments, a VR handset or other handheld device is in electronic communication with the VR headset 1104. The patient 1100 can provide inputs at the VR handset by gesturing with the VR handset or pressing buttons, bumpers, or joysticks on the VR handset. Optionally, the VR handset can include sensors that are configured to receive data from the patient (e.g., heart rate, skin conductance, etc.).

The VR headset 1104 and the VR method for evaluating patients for macular degeneration minimizes or eliminates many of the inconsistencies associated with the traditional method of evaluating macular degeneration, as described above with respect to FIG. 11A. First, the distance D2 between the eyes of the patient 1100 and the screens of the VR headset 1104 (where the visual task or test, such as the Amsler grid 1102, can be displayed), is constant because the VR headset 1104 is fixed to the patient's 1100 head. As a result, D2 is not only consistent among multiple patients, but it is also consistent throughout the evaluation of the patient 1100. Similarly, the screens of the VR headset 1104 are consistently at the same angle relative to the patient 1100 because the VR headset 1104 is secured to the patient's 1100 head. Thus, the angle A at which the patient 1100 holds the Amsler grid 1102 and the tilt of the patient's 1100 head do not affect the results of the evaluation. Finally, the VR headset 1104 allows the patient 1100 to more accurately respond to the visual task or test displayed on the screens. For example, where the screens of the VR headset 1104 display an Amsler grid (such as Amsler grid 1102 in FIG. 11A), the patient 1100 can use a VR handset to indicate which parts of the Amsler grid are missing, blurry, or wavy. Unlike in the traditional method, the VR method does not require the patient 1100 to change her perspective or move the Amsler grid when marking the Amsler grid because the VR method does not require the patient 1100 to use her hands to cover one of her eyes and hold the Amsler grid.

The VR method described herein is not limited to administering a single vision task or test on the screens of the VR headset. Using the VR headset and a software with an advanced processing algorithm and a broad library of vision tasks and tests, the VR method for detecting early signs of macular degeneration includes dynamic administration of visual tests on the screens of the VR headset and collecting the patient's responses to the various visual tests in order to evaluate the patient for macular degeneration.

After the patient dons the VR headset, the computing device causes a visual test to be administered on the screens of the VR headset. For macular degeneration, visual tests generally assess the color perception and acuity level of the patient. Examples of the visual test that can be administered are shown in FIGS. 11C-11D, which are described in greater detail below. The visual test can include a traditional Amsler grid, but it can also include variations of the Amsler grid. For example, the lines of the grid might be thicker or thinner, the squares of the grid might be larger or smaller, the grid might be shown at different contrast or brightness, etc. The visual test can also revolve around a different grid entirely. For example, the grid can have curved lines, the lines of the grid can be shown in different colors or a gradient of colors, the grid might be asymmetrical, the dot in the middle of the grid might be different sizes or colors, the dot can be in different parts of the grid, etc. The visual test can also constitute an array of dots or optotypes, where the sizes, the orientation, the color, the contrast or brightness, etc. can vary between visual tests or even throughout any given array.

In the preferred embodiment of the VR method for evaluating macular degeneration, the computing device causes the first visual test to be administered with the highest contrast and the highest brightness. The first visual test is usually the most basic version of the visual test (e.g., the standard Amsler grid, a symmetrical grid with lines that are easy to see, a symmetrical array of dots or optotypes, etc.), which is exemplified by FIGS. 11C-11D.

FIGS. 11C-11D each illustrate progression of visual tests that can be administered as part of the dynamic macular degeneration evaluation, in accordance with some embodiments disclosed herein. As shown in FIG. 11C, the first visual test 1106 is a standard Amsler grid (i.e., the most basic version of the visual test). Similarly, FIG. 11D shows that the first visual test 1116 is a basic array of evenly spaced, mid-sized dots.

When the first visual test is displayed on the screen of the VR headset, the patient responds. This response can include data (or involuntary responses) such as blinking, pupil dilation, response times, and other biodata (e.g., heart rate or skin conductance). The response can also include inputs (or voluntary responses) such as eye movements, descriptions of what the patient sees, or indications as to which parts of the first visual test appear distorted or missing. The input can be visual cues or verbal responses detected by the VR headset as well as gestures detected by the VR handset. The patient's response to the first visual test can be referred to as the first input, and the first input informs the computing device and/or the physician as to what parts of the first visual test the patient does and does not perceive.

After or while the VR headset collects the first input from the patient, the computing device processes the first input to determine the characteristics of the next visual test. In particular, the computing device analyzes the first input whether to make the next visual test more difficult, how difficult to make the next visual test, and what aspect(s) of the visual test should change (e.g., the contrast, the brightness, the size of optotypes, the thickness of lines, etc.) Optionally, the computing device also analyzes the patient's medical history when determining the characteristics of the next visual test. Upon determining the characteristics of the next visual test, the computing device causes the next visual test (or the second visual test) to be conducted on the screens of the VR headset.

In some embodiments, the VR headset (and, where applicable, the VR handset) collects the first input in real-time, while the patient responds to the first visual test. The real-time collection of the first input facilitates the dynamic nature of the VR method disclosed herein because the real-time collection aids the computing device in determining the characteristics of the second visual test as the patient responds to the first visual test. Because of this real-time collection, the computing device does not need to wait until the patient finishes the first visual test before causing the second visual test to start. Once the VR computing device gathers enough information from the first visual test, the computing device can move on and start gathering information from the second visual test. This dynamic testing facilitates faster macular degeneration evaluation. As a result, the patient can undergo a greater number of visual tests in a shorter period of time, as compared to traditional evaluation methods. This increased efficiency not only reduces the cost of the evaluation (by reducing the amount of time the physician has to spend with each patient), but it also allows for a deeper evaluation of the patient.

For example, turning to FIG. 11C, if a patient does not see any distortion when observing the first visual test 1106 in a traditional evaluation setting, the physician will note that the patient does not have any vision loss, determine that the patient does not have macular degeneration, and move on to discuss something else. However, with the dynamic evaluation method, the computing device might follow path 1108 and cause the second visual test 1110, which is more difficult, to be shown on the screens of the VR headset. The patient will respond to the second visual test 1110, and the VR headset will collect that response (which can also be referred to as the second input). The computing device can continue to conduct increasingly difficult visual tests (and the VR headset can continue to collect patient responses) until the computing device detects even the smallest amount of vision loss, which can help detect macular degeneration before the vision loss begins or even predict future macular degeneration. On the other hand, if a patient sees distortion when observing the first visual test 1106, the computing device might follow path 1112 and cause the alternative second visual test 1114, which is easier than the first visual test 1106, to be displayed on the screens of the VR headset. The computing device can continue to show easier visual tests (and the VR headset can continue to collect patient responses) until the computing device determines the fullest extent of the patient's vision loss. This information can be useful when prescribing treatment for the patient's macular degeneration and predicting future vision problems.

There are numerous characteristics of the visual test that the computing device can change in order to change the difficulty of the visual test. As described above with respect to FIG. 11C, the size of the squares and the thickness of the lines that make up an Amsler grid can be changed. Smaller squares and thinner lines are harder to perceive and require greater visual acuity, while larger squares and thicker lines are easier to perceive and require less visual acuity. FIG. 11D shows a similar example with an array of dots. In FIG. 11D, the first visual test 1116 is an array of mid-sized dots. If the patient accurately perceives the first visual test 1116, then the computing device follows path 1118 and causes the second visual test 1120 to be displayed on the screens of the VR headset. The second visual test 1120 is harder than the first visual test 1116 because the second visual test 1120 is an array of small dots that are more difficult to see and distinguish from each other. On the other hand, if the patient has trouble perceiving the first visual test 1116, then the computing device may follow path 1122 and cause the alternative second visual test 1124 to be displayed on the screens of the VR headset. The alternative second visual test 1124 is easier than the first visual test 1116 because the alternative second visual test 1124 is an array of large dots that are easier to see.

In some embodiments, the computing device can change the difficulty of the visual test by changing the brightness of the VR headset screens. The visual tests are harder to see when the screens are set to a lower brightness.

In other embodiments, the computing device can change the difficulty of the visual test by changing the contrast of the visual test. For example, where the visual test is an array of optotypes, the contrast between the negative, background space and the optotypes might be decreased, which makes the optotypes harder to see and increases the difficulty of the visual test.

In some embodiments, the visual test assesses the patient's ability to perceive colors, and the difficulty of the visual test is changed by adjusting the number of colors shown in the test and/or the saturation of the colors shown in the test. For example, the first visual test may show several lines with a first portion of the lines in red and a second portion of the lines in blue. If the patient correctly identifies where the color changes, the computing device might increase the difficulty of the test by making the lines change from red to blue more gradually. The computing device can also increase the difficulty by adding more portions of lines in different colors. In an example of adjusting the saturation of the colors, the first visual test may show a series of optotypes in green, and a second, more difficult visual test may show the optotypes in a less saturated green because less saturated colors are more difficult for the patient to perceive.

Other embodiments of the evaluation method may include the computing device changing the characteristics of the visual test by testing different areas of the retina. For example, the first visual test may be displayed in the center of the patient's line of vision, which tests the most active portions of the macula. Meanwhile, the second visual test may be displayed towards the edges of the patient's line of vision to test the mid-periphery of the macula.

Based on the inputs received in response to the visual tests, the computing device evaluates the patient for macular degeneration. The computing device evaluates the patient for macular degeneration by comparing the patient's medical history and the patient's responses to the visual tests to a database of responses from individuals with known visual health statuses (e.g., individuals without macular degeneration, with early-stage macular degeneration, with moderate macular degeneration, with severe macular degeneration, etc.). The computing device can compare any or all of the patient's responses to the database. In some embodiments, the computing device diagnoses the patient with macular degeneration and/or recommends treatment to reduce the symptoms of macular degeneration. In other embodiments, the computing device predicts the likelihood that the patient will develop macular degeneration in the future and recommends medication, lifestyle changes, etc. to reduce the patient's likelihood of developing macular degeneration.

In some embodiments, the computing device is electronically connected to a web portal and generates a report that is accessible via the web portal. In some embodiments, the computing device reports the patient's responses to the web portal in real-time during the administration of the visual tests. A physician can access these reports at the web portal and use this information to influence the direction of the evaluation (e.g., by changing characteristics of the visual tests or the frequency with which the visual test is changed). The physician can also use the information in the report to guide conversations with the patient, which can help the physician diagnose the patient with macular degeneration or other ocular conditions.

Once a patient is diagnosed with macular degeneration, it can be valuable to assess the macular degeneration of each eye because the macula of each eye can deteriorate at different rates. In traditional optometric settings, a physician might show a patient an object, such as a red ball, and ask the patient to look at the red ball with her left eye covered and then with her right eye covered. After the patient observes the red ball with each eye individually, the physician asks the patient to describe the difference in the color perceived by each eye and use the patient's verbal description to assess the rate at which each of the patient's eyes is deteriorating. However, this traditional evaluation is highly subjective and, thus, inaccurate.

Evaluating patients using VR makes an accurate assessment of the degree or rate of macular degeneration in each eye possible. When the patient dons the VR headset, the computing device causes a first image to be displayed on the left screen of the VR headset and a second image to be displayed on the right screen of the VR headset. The first and second images are distinct images such that the patient's eyes will not easily combine the first and second images. The first and second images can be one or more of environments, scenes, optotypes, photographs, etc. In some embodiments, the first and second images are displayed with one on top of the other. The first and second images can be displayed such that the patient's eyes do not merge the first and second images into a single image.

Upon viewing the first and second images, the patient responds to the first and second images individually. The response can include the patient's involuntary responses, and the VR headset will collect data such as the patient's response time, blink rate, pupil dilation and biodata (e.g., heart rate, skin conductance, and the like). The response can also include the patient's voluntary responses, and the VR headset will collect input such as the patient's eye movements, verbal statements, or hand gestures. The patient's responses (which can include both data and input) to the first and second images can be referred to as the first input and the second input, respectively. In some embodiments, the VR headset collects the first and second input in real-time as the patient perceives the first and second images.

Where the patient's maculae are deteriorating at different rates, the patient will perceive the first and second images differently. For example, the patient might perceive the first image as brighter, more contrasted, more saturated, sharper, etc. than the second image. Through the VR headset, the patient will be prompted to adjust the first and second images until they have the same or similar brightness, contrast, saturation, acuity, etc. An example of this is shown in FIGS. 11E-11F.

FIGS. 11E-11F illustrate a VR headset with different images being shown on each screen so that a patient can individually adjust the contrast of each image until the images have the same contrast, in accordance with some embodiments described herein. FIG. 11E shows a headset 1104 with a first screen 1126 and a second screen 1128. The first screen 1126 shows a first image 1130 of a trumpet, and the second screen 1128 shows a second image 1132 of a plant. The first and second images 1130, 1132 are faded gray, with the first image 1130 being a more faded gray than the second image 1132 to represent that the patient perceives the first image 1130 as having a lower contrast than the second image 1132. The VR headset 1104 may prompt the patient to adjust the first image 1130 until the first image 1130 is the same contrast as the second image 1132. The VR headset 1104 may also prompt the patient to adjust the first image 1130 and the second image 1132 until both the first and second images 1130, 1132 appear at full contrast (in this situation, appear black) as shown in FIG. 11F.

The computing device receives the patient's adjustments and calculates the degree of macular degeneration in each of the patient's eyes by inversely correlating those adjustments to the degree of macular degeneration. For example, if the patient had to increase the contrast of the first image 1130 by 60% and the patient had to increase the contrast of the second image 1132 by 30%, then the computing device will process these adjustments and calculate that the macula of the first eye is deteriorated 30% more than the macula of the second eye.

The computing device can process these calculations (i.e., the degree of macular degeneration) to recommend treatment or further evaluations to address the patient's macular degeneration. In some embodiments, processing the calculations includes implementing an advanced algorithm to compare the degree of macular degeneration to a database with information from individuals with known degrees of macular degeneration. In other embodiments, processing the calculations includes analyzing the patient's medical history in conjunction with assessing the patient's current degree of macular degeneration in each eye.

Optionally, the computing device generates a report with this information, and this information can be accessed by the patient or by the physician at the web portal described above.

### Examining Tear Film Quality Using High-resolution Imagery

The eyes are coated with a tear film that is made up of a three-layered structure: a base layer made of mucus, an aqueous layer, and a lipid layer. The mucus layer makes the surface of the eye hydrophilic, the aqueous layer provides tears and flushes debris out of the eyes, and the lipid layer prevents evaporation of the tears and gives the eye an optically smooth surface. A tear film is formed when a person blinks. As the tears are exposed to air, the tears begin to evaporate, and the tear film breaks up. The breakup of the tear film induces the person to blink, which forms a new tear film. Good quality tear film is an important aspect of both clear vision and eye comfort.

Traditionally, tear film quality is assessed by using a paper strip or a cotton thread to collect a sample of a patient's tear film or apply dye to the patient's tear film. These traditional methods facilitate a chemical analysis of the tear film and make the tear film easier to observe. However, the traditional methods are very invasive in that they require a physician to touch the patient's eyeball. Contact with the patient's eyeball induces excessive blinking and, thus, excessive tear film production. These complications limit the repeatability and the accuracy of the test results received from the traditional methods. Through the use of a VR system, a less-invasive method for assessing tear film quality is possible.

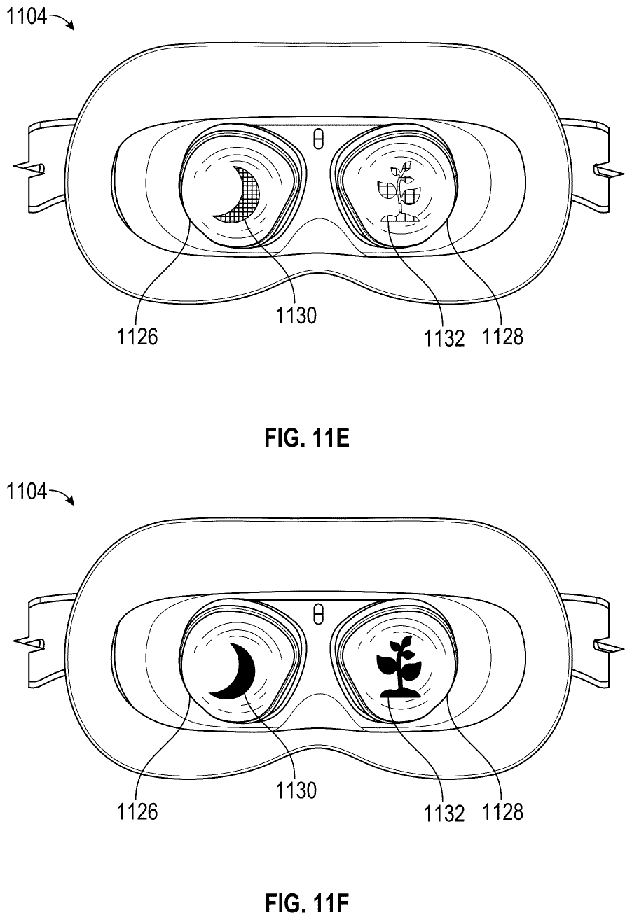

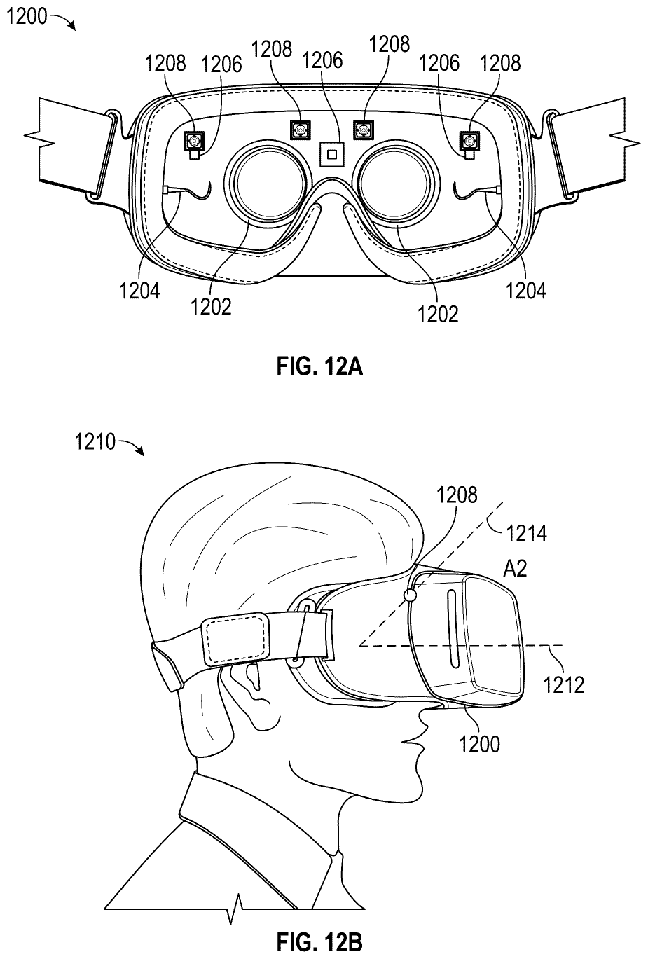

FIG. 12A illustrates a VR system for examining tear film quality using high-resolution imagery, in accordance with some embodiments disclosed herein. The VR system comprises a VR headset 1202 that is electronically connected to a computing device. The VR headset 1202 may include screens 1202, probes 1204, sensors 1206, and cameras 1208. The screens 1202 are configured to display different images, fixation targets, and other visual stimuli for a patient wearing the VR headset 1200.

The probes 1204 are configured to collect samples of tear film from the patient's eye. Each probe 1204 has a tip that protrudes from the VR headset 1200. The probe 1204 can protrude from any portion of the VR headset 1200 and is not limited by the position displayed in FIG. 12A. For example, in some embodiments of the VR headset 1200, there is only one probe 1204, and it is positioned centrally between the two screens 1202.

The tip of the probe 1204 is a C-shaped hook or a curved tip, where the curved or bent portion of the tip is configured to contact the patient's eye. The probe is positioned within the VR headset 1200 such that the curved portion of the tip is within 1 mm of the outer portion of the patient's eyelid when the probe 1204 is not in use. In a preferred embodiment, the curved portion of the tip is within 0.5 mm of the outer portion of the patient's eyelid when the probe 1204 is not in use. In some embodiments, the curved potion of the tip is within 0.3 mm of the outer portion of the patient's eyelid when the probe 1204 is not in use. When the computing device instructs the VR headset to collect a tear film sample from the patient's eye, the probe moves from its default position towards the patient's eye until the curved portion of the C-shaped tip contacts the surface of the patient's eye and collects a sample from the patient's tear film.

The VR headset 1200 also includes sensors 1206 that are configured to analyze the sample of tear film collected by the probes 1204. The sensors 1206 can measure one or more of the amount of salinity in the tear film, the chemical composition of the tear film, and the amount of particulate matter in the tear film. The sensors 1206 can be arranged throughout various portions of the VR headset 1200 and are not limited to the arrangement shown in FIG. 12A. In some embodiments, the sensors 1206 are embedded on the probe(s) 1204. In other embodiments, the sensors 1206 may be constructed and operable in accordance with any variety of conventional technologies.

The VR headset 1200 also includes cameras 1208 that are configured to capture high-resolution images of the tear film. These high-resolution images are processed by the computing device to identify one or more of the thicknesses of the tear film and each of its layers, the amount of particulate matter in the tear film, and tear film breakup patterns. The VR headset 1200 has four cameras 1208, with two cameras 1208 pointed at each eye. Each camera 1208 is coupled with infrared illuminators to ensure clear imaging under various lighting conditions.

The cameras 1208 have lenses that are specially designed to take high-resolution photographs of the tear film. The lenses have a diameter of approximately 15 mm. In some embodiments, the diameter of the lens is 14.2 mm or less. As a result of the small lens, the camera 1208 can take photographs of the tear film with a magnification of at least 6×, at least 7×, at least 8×, or at least 9×. In some embodiments, the camera 1208 can take photographs of the tear film with a magnification of at least 5×, 6×, 7×, 8×, or 9×. The lens is fabricated using a procedure similar to the procedure for fabricating gas permeable contact lenses with a key difference being the speed at which the lathe rotates to shape the lens and cut the lens to the necessary diameter and thinness. The lathe rotates 80% slower to produce the camera lens than a lathe would typically rotate to produce a contact lens. In some embodiments, the lathe rotates 90% slower. In other embodiments, the lathe rotates 92% slower.

FIG. 12B illustrates the position of the cameras on the VR headset relative to the patient's eyes, in accordance with some embodiments described herein. The patient 1210 wears a VR headset 1200. The position of the cameras 1208 can be described in relation to a transverse plane 1212 of the patient's 1210 eyes. The cameras 1208 are positioned on a plane 1214, where the angle between the plane 1214 and the transverse plane 1212 is angle A2. A2 is between 30 degrees and 60 degrees. In some embodiments, A2 is between 37 degrees and 53 degrees. In a preferred embodiment, A2 is approximately 45 degrees.

To begin the VR tear film quality evaluation, the patient dons the VR headset. The computing device causes a variety of visual stimuli to be displayed on the screens of the VR headset. The visual stimuli induce or guide the patient to keep his eyes open. As a result, the patient will not blink and form a new tear film, which makes it possible for the VR headset to observe the patient's tear film. Delaying the prevention of a new tear film makes it possible for the VR headset to observe the patient's tear breakup patterns and determine the patient's tear breakup time (the amount of time it takes for the tear film to dry up, at which point the eyes will blink to produce a new tear film). A good quality tear film lasts approximately 10 to 35 seconds. Tear films with breakup times of less than 10 seconds are typically indicative of tear film instability, at which point the patient may experience eye dryness and discomfort.

In some embodiments, the method for assessing tear film quality includes adjusting the characteristics (e.g., brightness, contrast, saturation, level of motion, etc.) of the visual stimuli. For example, if the visual stimuli are too bright, the patient might have trouble keeping his eyes open, which can cause discomfort and reduce the accuracy of the tear film evaluation because it prevents the VR headset from observing the patient's natural tear breakup patterns. The cameras and/or sensors observe the patient's blink rate, and if the blink rate is too high, the computing device automatically adjusts the characteristics of the visual stimuli to help the patient keep his eyes open. This feedback loop helps increase the accuracy VR tear quality assessment.

The computing device also causes the probe (or probes) of the VR headset to move out of its default position and towards the patient's eyes to collect a sample of the patient's tear film. The sensors, embedded on the VR headset or on the probe, analyze the tear film sample to identify the amount of salinity, the chemical composition, and/or the amount of particulate matter in the tear film. In some embodiments, the sensors analyze the patient's tear film in real time as the patient perceives and reacts to the visual stimuli.

Moreover, the computing device causes the cameras to capture high-resolution images of the patient's tear film before, during, and after the display of the visual stimuli. The cameras capture images of the tear film to identify the amount of particulate matter in the tear film and to gather information about the tear film dynamics (e.g., tear breakup time, lipid layer thickness, tear stability, tear breakup patterns, etc.). In some embodiments, the cameras continuously capture images of the patient's tear film throughout the evaluation, and the computing device processes the images in real-time to generate a report.

Traditionally, dyes or stains are applied to the patient's eyes as part of the tear film evaluation. However, applying dyes or stains directly to the ocular surface can irritate the patient's eyes and change the characteristics of the tear film. For example, in the traditional method, fluorescein (which has an orange color) is applied to the ocular surface, and a physician observes the tear film under a blue light and/or with a yellow filter. Touching the patient's eye to apply the fluorescein induces reflex tearing and reduces the surface tension of the tear film, which means the physician cannot observe the patient's natural tear film dynamics. Moreover, applying the fluorescein floods the patient's eye with liquid, which swamps the normal tear film.

By capturing images of the tear film as different visual stimuli are displayed on the screen, the VR method for tear film evaluation disclosed herein makes it possible to gather information from the traditional dyeing process in a less invasive manner. This is because the visual stimuli on the screens can reflect different colors of light onto the patient's eyes. For example, an orange or yellow light can highlight epithelial cell loss and a green or reddish-pink light can highlight dead cells. With the VR method of the present application, it is possible to observe the tear film characteristics highlighted by different colored lights (the equivalent of dyes in the traditional method) without disturbing the patient's natural tear film quality and patterns. Overall, this increases the accuracy and usefulness of the tear film quality evaluation.

The cameras can also be used to film the patient's blinking patterns, which is also indicative of the patient's tear film quality. A typical blink pattern, which correlates with a healthy tear film, is approximately one blink every five seconds, or approximately 11 blinks per minute.

Additionally, a comprehensive tear film assessment includes evaluation of anatomical structures that surround the eye. Thus, the cameras can also be used to film or capture images of the tear ducts, the eyelids, the muscles surrounding the eyes, etc. Irregularities discovered within these structures can also be useful in assessing the patient's tear film quality.

After the VR headset collects the tear film sample and captures images and/or videos of the tear film, the computing device receives that data and compares the sample, images, and/or videos to a database to evaluate the patient's tear film. The database is composed of tear film data from individuals with known tear film qualities. In some embodiments, the computing device compares the patient's data to the database in real-time as the VR headset continues to collect data.

After or as the computing device processes the patient's data, the computing device generates a report. The report contains information such as the ratio of the lipid layer to the aqueous layer, the amount of particulate matter on the tear film, the tear film osmolarity, etc. The report can also include recommendations for treatment (e.g., eye drops, medication, lifestyle changes, training blinking patterns, etc.).

In addition to assessing the tear film quality, the VR headset can be used to implement a method for improving tear film quality by training the patient's blinking patterns. For example, the patient may need to be taught how to blink more often so that his eyes are more often protected by a new, clean tear film. This method includes conducting a visual task on the screens of the VR headset that induces the eyes of the patient to move up and down at a specific rate. When the eyes move up and down, the eyelids will naturally follow, which induces blinking and the production of a new tear film. In some embodiments, the visual task is a dot moving up and down, where the patient is prompted to follow the dot. In other embodiments, the visual task is a pair of virtual eyes blinking at the appropriate rate, and the patient is prompted to mirror the virtual eyes. Optionally, the visual task is a semicircle filled with a horizontal gradient, where the diameter of the semicircle is at the bottom of the screen. The colors gradient translates up and down throughout the visual task, and the patient is prompted to follow one of the colors of the gradient. Other visual tasks are also possible.

### Diagnosing and Monitoring Progression of Retinal Disorders With Focused Vision Tests

Retinal exams are a valuable component of vision health. Not only do retinal exams help detect eye diseases such as macular degeneration and glaucoma, but, for people with diabetes, retinal exams also help track damage to blood vessels in the retina, which can lead to vision loss. Retinal exams are used to observe the blood vessels in the eye, the optic nerve, the macula, and the retina itself. A traditional retinal exam starts by measuring the patient's visual acuity, which can include measuring the patient's eye pressure as well as observing the patient's pupils. Next, a physician will apply eye drops to the patient's eyes to dilate her pupils. Once the pupils are dilated, which can occur about 20-30 minutes after the eye drops are applied, the physician shines lights into the patient's eyes to view the back of her eyes. Not only is this process uncomfortable for the patient (due to the eye drops and the lights), but this process is also time consuming. Disclosed herein are VR systems, headsets, and methods for conducting less invasive, faster, and more comprehensive retinal exams.

A VR system for diagnosing health conditions related to the retina and monitoring the progression of those health conditions includes a VR headset in electronic communication to a computing device. The VR system is portable and user-friendly, which makes the VR system convenient to use in both clinical and at-home settings. The use of the VR system in homecare settings is particularly valuable for monitoring the progression of retinal disorders because a patient can use the VR system on her own in the comfort of her own home on a daily or weekly basis, as instructed by her physician, to keep track of the baseline status of her optic nerve, macula, retina, etc.

The VR headset includes sensors, cameras, screens, and one or more probes. This VR headset is similar to the VR headset 1200 described above with respect to FIGS. 12A and 12B. The sensors of the VR headset are configured to scan the blood vessels in the patient's eyes as well as her optic nerves, maculae, and retinas. In a preferred embodiment, the sensors are infrared sensors. Optionally, the sensors each have a pixel density of 10 μm or less. In some embodiments, this pixel density is approximately 8 μm or less. In other embodiments, this pixel density is approximately 6 μm or less.

The cameras of the VR headset are configured to scan and take high-resolution images of the patient's blood vessels, optic nerves, maculae, and retinas. The cameras of this VR headset are akin to the cameras 1208 of VR headset 1200 described above with respect to FIGS. 12A and 12B. This means that some embodiments of this VR headset have infrared cameras. Similarly, this means that in a preferred embodiment, the VR headset has two cameras pointed at each of the patient's eyes, and each camera is positioned at an angle that is 45 degrees above a transverse plane of the patient's eye.

The screens of the VR headset are configured to display vision tests for the patient. The screens can also be used to project light onto the patient's eyes while the sensors scan or while the cameras capture or record.

The probe or probes of the VR headset are configured to detect retinal markers on the patient's retina. In some embodiments, the probe comprises ultrasound sensors.

To begin a VR retinal exam, the patient dons the VR headset. The computing device instructs the sensors and cameras to conduct a scan of the retinal blood vessels, optic nerves, maculae, and retinas, which will be referred to as “the eyes” for the purposes of this description. The scan comprises a 360-degree scan of each eye that can start and end at the patient's optic nerve. Scanning the eyes gathers data about the patient's retinal function, which includes monitoring the patient's eye movements, fixation stability, and response times (to the vision tests displayed on the screens of the VR headset, described in greater detail below).

The cameras can also take images and/or videos of the eyes during the scans. The cameras capture high-resolution images and/or videos that can be processed by the computing device or analyzed by the patient's physician. In particular, the cameras can capture images and/or videos of areas of the eyes that show signs of retinal disorders for further processing and/or analysis.

The scans, images, and videos can be used to identify areas of the eyes that show signs of retinal disorders, which will be referred to as “target areas” for the purposes of this description. Examples of target areas include scarring from diseases and detachment from autoimmune inflammation. The sensors and cameras can track ocular movements with sub-millimeter accuracy, which means that the VR headset can identify even the smallest target areas. This facilitates very targeted evaluations, which leads to early detection of retinal disorders, nuanced diagnoses, and precise monitoring of the progression of retinal disorders. In some embodiments, the sensors and cameras can identify movements that are smaller than approximately 0.5 mm. In other embodiments, the sensors and cameras can identify ocular movements that are smaller than approximately 0.3 mm.

Once the VR headset conducts a first scan and the computing device identifies the first target area, the computing device isolates the first target area of the eyes and focuses the evaluation on the first target area. In particular, the computing device processes the data obtained from the scan, the images, or the videos and chooses a first vision test to administer to evaluate the first target area.

The vision tests include acuity tests, color perception tests, visual field tests, etc. For example, the vision test may include an array of letters or other optotypes, such as a 5×5 array of “Cs.” The vision test may also include a display of a building that extends through both the patient's central and peripheral vision. Additionally, the vision tests can be both dynamic and static. Dynamic and static stimuli activate different parts of the retina, which is useful for evaluating the target areas. For example, a first vision test that is dynamic (e.g., a moving pattern of black and white lines) might activate the first target area, while a second vision test that is static (e.g., a single optotype on the screen that changes from a “C” to an “E”) might activate a second target area.

While the first vision test is displayed on the screens of the VR headset, the patient responds to the first vision test with involuntary responses (such as changes to heart rate, blink rate, pupil dilation, eye pressure, skin conductance, reaction times, etc.) and voluntary responses (such as eye movements, verbal descriptions, hand gestures (where the VR system includes a VR handheld device in electronic communication with the VR headset and the computing device), etc.). While the patient provides responses to the first vision test, the probe of the VR headset searches for and identifies retinal markers (referred to as “the first retinal markers”) in the first target area. The probe searches for visual perception changes in order to detect disorders such as diabetes, glaucoma, and even Alzheimer's using the retinal markers. In some embodiments, the probe is an ultrasound sensor with a very fine resolution, such that the ultrasound sensor can detect subtle changes on the retina and isolating retinal markers. The probe detects retinal markers by transmitting ultrasound signals towards the eyes and receiving the signals that are reflected.

Optionally, the VR headset probes the patient's eyes in real-time as she responds to the first vision test. The real-time detection of first retinal markers facilitates the dynamic nature of the VR method disclosed herein by giving the computing device the information it needs to isolate a second target area and determine the characteristics of a second vision test. Because the probing occurs in real-time, the computing device does not need to wait until the patient finishes the first vision test before causing the second vision test to start. Once the VR headset collects enough information about the patient's response to the first vision tests and the computing device sufficiently assesses the first target area, the computing device can cause the second vision test to be displayed on the screens of the VR headset and instruct the VR headset to start collecting information about the patient's responses to the second vision test. As a result, the VR system can assess more target areas and administer more vision tests in a shorter period of time, as compared to traditional evaluation methods. This increased efficiency reduces the amount of time that the physician has to spend with the patient (which reduces the cost of the evaluation), and the quantity of testing that is possible from this efficiency allows for a more comprehensive and nuanced evaluation of the patient's retinal blood vessels, optic nerve, macula, and retina.

Moreover, while the patient responds to the first vision test, the computing device instructs the VR headset to conduct a second scan of the retina. As with the first scan, the sensors and the cameras of the VR headset do a 360-degree scan of the patient's eyes (i.e., retinal blood vessels, optic nerves, maculae, and retinas) to gather data about the patient's retinal function.

Conducting a second scan of the patient's eyes while the patient responds to the first vision test is valuable because the second scan provides different data to the computing device than the first scan does. Likewise, conducting third or fourth scans while the patient responds to second or third vision tests is also valuable. This is because the different vision tests are directed to different target areas and stimulate different parts of the retina. For example, dynamic and static stimuli activate different parts of the eyes and elicit different reactions, so a scan of the eyes during a dynamic vision test provides different information than a scan of the eyes during a static vision test. Moreover, different vision tests induce different changes to the retinal surface, such as depth changes, pigmentary changes, color changes, etc. Overall, repeatedly conducting scans during the different vision tests provides more information to the computing device, which makes the evaluation more accurate.

The computing device processes the data gathered during the scan, as well as the photographs and videos taken by the cameras during the scan, to isolate a second target area, which will be the focus of a second vision test.

In some embodiments, the VR headset communicates the second scan data to the computing device in real-time as the patient responds to the first vision test. Optionally, the computing device also processes the scan data to identify and isolate the second target area in real-time. As with the real-time detection of retinal markers by the probes on the VR headset, described above, real-time scanning of the eye during the first vision test facilitates the dynamic nature of the VR method because the computing device can dynamically switch between vision tests that are focused on different target areas. Dynamically switching between tests increases the efficiency and comprehensiveness of the retinal evaluation, as compared to traditional retinal exams. The dynamic nature of the VR method for monitoring and detecting retinal disorders makes it possible to detect retinal disorders in early stages and identify changes (including small, nuanced changes) in the progression of the patient's existing retinal disorders. This information is vital for determining how to treat retinal disorders and preserve the patient's vision.

When the computing device processes the second scan data and isolates the second target area of the retina, the computing device determines the characteristics of the second vision test. In some embodiments, this includes simply adjusting the characteristics of the first vision test.

Optionally, the VR headset collects the patient's responses to the vision tests. For example, the sensors and cameras might collect the patient's blink rate, reaction times, eye movements, etc. and communicate that information to the computing device. The computing device processes that information by comparing the information to a database of responses from individuals with a variety of known retinal conditions or ocular disorders.

The computing device causes the second vision test to be displayed on the screens of the VR headset and instructs the probes of the VR headset to detect a second set of retinal markers in the second target area. In some embodiments, the computing device processes the data about the retinal markers (such as location, severity, etc.) by comparing the retinal markers to a database of retinal markers from individuals with a variety of known retinal conditions, or even from individuals with other ocular disorders.

Moreover, the computing device instructs the sensors and cameras of the VR headset to conduct a third scan of the patient's eyes. Optionally, the computing device processes the scan data by comparing it to a database of retinal scan data from individuals with a variety of known retinal conditions or ocular disorders.

Where the VR method includes comparing the response information, the retinal markers, and/or the retinal scan data to a database, the computing device can be used to identify deviations that indicate retinal pathology such as Stargardt disease, hypertension, or diabetes.

### Assessing Rapid Eye Movement Patterns Using Virtual Reality

There are two broad categories of sleep: rapid eye movement (REM) sleep and non-REM (NREM) sleep. There are three stages of NREM sleep: stage 1 drifting off or light sleep, stage 2 light sleep, and stage 3 deep sleep. Each stage of sleep is distinct and serves a different purpose. In stage 1, people's brain waves, heart rate, breathing, and eye movements slow down right after they fall asleep. In stage 2, people's brain waves continue to slow down, and they experience short, strong bursts of electrical activity in their brains called sleep spindles, during which brains organize memories and information. The deepest sleep occurs in stage 3. In stage 3, the body repairs injuries and reinforces the immune system. Sometimes, sleep spindles also occur in stage 3. Stage 3 is followed by REM sleep. During REM sleep, people experience the most dreams, their brain activity looks very similar to their brain activity when they are awake, and their eyes move rapidly behind their eyelids. REM sleep is a key aspect of developing cognitive skills, transferring short-term memories into long-term memories, and processing emotional experiences. When people get insufficient REM sleep, they may have difficulty remembering things, have trouble regulating their emotions, have trouble concentrating, and even have weakened immune systems.

Observing a person's REM patterns provides valuable information about a person's sleep quality and neurological function. Identifying discrepancies in REM patterns (e.g., excessive REM sleep or inadequate REM sleep) can be indicative of sleep disorders such as narcolepsy, sleep apnea, and REM sleep behavior disorder. Discrepancies in REM patterns can also correlate with certain ocular conditions.

Traditionally, REM sleep is observed through sleep studies. To participate in a traditional sleep study, a person must spend a night in a clinic or doctor's office. Healthcare providers attach sensors to various parts of the person's body and monitor the person's breathing rates and eye movements while he sleeps. Because he is sleeping in an unfamiliar environment with various sensors and wires attached to him, the person is significantly less likely to sleep well during the sleep study. Additionally, sleep studies are inconvenient for the person participating and can be very expensive, which means that multiple sleep studies are not an option for most people.

Using a VR system and a VR method for assessing sleep patterns, a person can not only conduct their own VR sleep study in the comfort of his own home, but he can also conduct VR sleep studies on a regular basis. This provides the person with a larger and more accurate dataset, which can help diagnose sleep disorders and other ocular conditions that correlate with discrepancies in REM patterns. Moreover, periodic VR sleep studies can be used to track REM patterns overtime, which makes it possible to track the progression of REM patterns and identify nuanced changes to sleep patterns that may be indicative of larger issues.

A VR system for assessing REM patterns includes a VR headset in electronic communication with a computing device. The VR system is portable and user-friendly, which makes the VR system convenient to use in both clinical and at-home settings. The use of the VR system in homecare settings is particularly valuable because it facilitates regular sleep studies in the comfort of the person's own home.

The VR headset includes cameras, which are configured to track the eye movements of the patient while the patient is sleeping. The cameras of this VR headset are akin to the cameras 1208 of VR headset 1200 described above with respect to FIGS. 12A and 12B. This means that in a preferred embodiment, the VR headset has two cameras pointed at each of the patient's eyes, and each camera is positioned at an angle that is 45 degrees above a transverse plane of the patient's eye. Moreover, in some embodiments, the cameras are infrared cameras, which increase the resolution of the images and videos captured by 80%, as compared to non-infrared cameras.

The VR headset also includes sensors configured to track the eye movements of the patient while the patient is sleeping. In some embodiments, the sensors are infrared sensors. The sensors may be constructed and operable in accordance with any variety of conventional technologies.

Pairing the sensors with the cameras increases the resolution of the tracking by 15-20%, as compared to only using cameras to track the patient's eye movements. The sensors and cameras are specially configured to track rapid eye movements, which means they can track even the smallest eye movements. In some embodiments, the sensors and cameras can track eye movements with a margin of error of 2 mm or less. In other embodiments, the sensors and cameras can track eye movements with a margin of error of 1.5 mm or less. Optionally, the sensors and cameras can track eye movements with a margin of error of 1 mm or less.

The computing device is configured to process the data collected by the VR headset. In some embodiments, the computing device processes the data in real-time as the VR headset collects the data.

The computing device can also include a user interface, at which the patient can run his own sleep monitoring sessions. The user interface can also be used by a physician in clinical settings, as needed. The user interface comprises a portal for accessing the sleep monitoring sessions during and after the sleep monitoring sessions. The user interface can also display a report generated by the computing device, where the report includes the data collected during the VR sleep study as well as potential diagnoses, recommendations for further evaluations, and recommendations for treatment or lifestyle adjustments.

The VR method for conducting sleep studies begins when the person dons the VR headset. The person falls asleep, and the sensors and cameras of the VR headset track his eye movements while he sleeps. The sensors and cameras can also track the person's breathing patterns and heart rate while he sleeps.

In some embodiments, the sensors and cameras continuously track the person's eye movements. In other embodiments, the sensors and cameras are only triggered to track the eye movements periodically—for example, when the eye movements become more rapid or when the person's breathing patterns change.

This data is collected by the VR headset and sent to the computing device. Once the computing device receives the data, the computing device processes the data to distinguish between the person's different sleep stages, which helps the computing device identify the person's REM periods. In some embodiments, the data is sent to and processed by the computing device in real-time as the VR headset collects the data. Optionally, where the data is received by the computing device and analyzed in real-time, the computing device can instruct the sensors and the cameras to adjust the focus of the eye tracking to target different portions of the patient's eyes.

The computing device can also generate a report using the data collected by the VR headset. This report can be accessed at the user interface of the computing device. The report can include a summary of the data collected by the VR headset as well as the results of the analysis conducted by the computing device. For example, the report can diagnose the person with sleep disorders or sleep-related ocular disorders. The report can also recommend that the person seek evaluations by a physician for sleep disorders or sleep-related ocular disorders. The report can also generally provide insights as to the person's overall eye health by providing information about the person's memory, cognition, perception, etc. Some reports might also include recommendations or advice about changes to the person's sleep habits that will help the person get the proper amount of REM sleep or generally improve the person's quality of sleep.

In some embodiments, the computing device generates the report in real-time as the VR headset collects the eye movement data and other biodata. Optionally, the computing device can generate the report 60 or fewer ms after processing the data. Optionally, the computing device can generate the report 50 or fewer ms after processing the data.

### Identifying and Categorizing Floaters in Simulated Lighting Conditions

As people age, strands of their vitreous stick together and cast shadows on their retinas. As a result, people see spots, squiggly lines, or threads floating across their vision. These are called floaters. Some floaters are temporary and harmless. However, floaters that are more severe can be indicative of serious eye conditions such as infections, injuries, uveitis, ocular bleeding, vitreous detachment, retinal tear, and retinal detachment—to name a few. For this reason, physicians will often ask about or look for floaters during eye examinations.

Traditionally, physicians ask patients to describe their floaters and then check patients' eyes for floaters by dilating their pupils with eyedrops and looking for floaters on the patients' eyes. The conversational aspect of the traditional examination is highly subjective because a lot of information can be lost between a patient's explanation and a physician's understanding. Moreover, the physician may not be able to identify all of the floaters that the patient describes because floaters are so small. This is exacerbated by the fact that the physician examines the eyes by looking for the floater itself while the patient sees the shadow of the floater, which is much larger than the floater. Consequently, the physician will be unable to observe the full scope of the floaters perceived by the patient. Disclosed herein are VR systems, headsets, and methods through which the patient can objectively show the physician all of the floaters she experiences so that the physician has a thorough understanding of the patient's floaters and can accurately evaluate the patient for eye conditions that correlate with floaters.

A VR system for identifying and categorizing floaters in various lighting conditions includes a VR headset in electronic communication with a computing device. In some embodiments, the computing device includes a user interface at which the physician or the patient can access the data collected by the VR headset (e.g., eye movements, blink rates, etc.) and the inputs provided by the patient (i.e., the creation of VR floaters, described below). The user interface can also be used by the physician to monitor the process and change aspects of the evaluation (e.g., change the lighting conditions or the VR environment).

The VR headset includes screens, sensors, cameras, and microphones. The screens of the VR headset are configured to display an environment with various lighting conditions, as controlled by the computing device. This is valuable because the intensity with which floaters appear depends on the lighting in the environment. For example, a patient may notice floaters more when they are outside during the daytime or in a brightly lit room. The patient interacts with the environment displayed on the screens by placing, sizing, and otherwise characterizing VR floaters in the VR environment that simulate the floaters she normally perceives (i.e., the floaters she perceives outside of the VR environment). Moreover, the three-dimensional environment displayed by the VR headset facilitates accurate placement, sizing, and characterization of the floaters in three-dimensional space, which makes the resulting ocular evaluation more accurate, as compared to the traditional method or even using a flat screen to display the floaters.

The sensors of the VR headset are configured to monitor the patient's eye movements as the lighting conditions of the environment change and as the patient creates VR floaters in the VR environment, as described above. In some embodiments, the sensors are infrared sensors. In other embodiments, the sensors each have a pixel density of 10 μm or less. Optionally, this pixel density is approximately 8 μm or less, or even approximately 6 μm or less. The sensors may be constructed and operable in accordance with any variety of conventional technologies.

The cameras of the VR headset are configured to monitor the patient's eye movements as the lighting conditions of the environment change and as the patient creates VR floaters in the VR environment. The cameras of this VR headset are akin to the cameras 1208 of VR headset 1200 described above with respect to FIGS. 12A and 12B. This means that some embodiments of this VR headset have infrared cameras. Similarly, this means that in a preferred embodiment, the VR headset has two cameras pointed at each of the patient's eyes, and each camera is positioned at an angle that is 45 degrees above a transverse plane of the patient's eye.

The microphone of the VR headset is configured to receive verbal inputs from the patient, such as verbal descriptions that supplement the creation of the VR floaters. In some embodiments, the microphone can also be used to track breathing patterns.

In some embodiments, a handheld device is electronically coupled to the VR headset and the computing device. The handheld device can be used by the patient to control the placement, size, and other characteristics of the VR floaters displayed in the VR environment.

The VR system is portable and user-friendly, which makes the VR system convenient to use in both clinical and at-home settings. The use of the VR system in homecare settings is particularly valuable for monitoring how the patient's floaters change and intensify because a patient can use the VR system on her own in the comfort of her own home on a daily or weekly basis, as instructed by her physician.

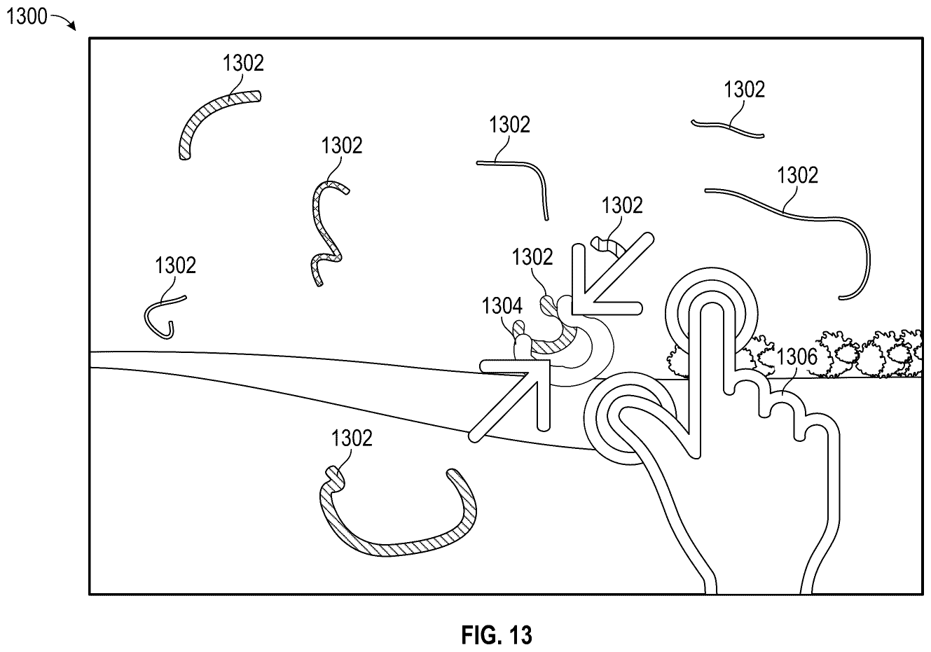

The VR method for identifying and categorizing floaters in various lighting conditions is a guided process through which the patient is prompted to create VR floaters so that the computing device or the physician can evaluate the patient's floaters and determine whether the floaters are harmless or whether the floaters are indicative of more serious ocular conditions. When the patient dons the VR headset, the computing device generates an environment on the screens of the VR headset, where the environment has a first lighting condition. The sensors and cameras monitor the patient's eye movements as she reacts to the environment being displayed. Next, the computing device prompts the patient (e.g., via text on the screen or audio instructions emitted on speakers of the VR headset) to describe or create VR floaters. The patient can then use verbal descriptions and/or hand gestures to create VR floaters. The use of hand gestures to create VR floaters is depicted in FIG. 13.

FIG. 13 illustrates a VR environment in which the patient can create VR floaters, in accordance with some embodiments described herein. The VR environment 1300 is displayed on the screens of the VR headset. The patient perceives real floaters 1302 in the VR environment, as she normally does outside the VR environment 1300. The patient creates VR floater 1304 and uses the VR hand 1306 to change the characteristics of the VR floater 1304 to match the characteristics of the real floater 1302. Changing the characteristics of the VR floater 1304 includes choosing the shape of the VR floater 1304, the thickness or size of the VR floater 1304, the transparency of the VR floater 1304, the location of the VR floater 1304, and even how the VR floater 1304 moves across the patient's vision. The patient can also use the VR hand 1306 to align the VR floater 1304 with the real floater 1302 she normally perceives outside of the VR environment 1300. Aligning the VR floater 1304 in three-dimensional space in the VR environment 1300 provides an objective description of the patient's real floaters 1302, which can be used by the computing device or the physician to evaluate the patient.

The VR headset collects inputs from the patient about the placement, size, characteristics, density, depth, etc. of the floaters and communicates those inputs to the computing device. In some embodiments, the VR headset communicates the inputs to the computing device in real-time as the patient provides the inputs.

Upon receiving the inputs, the computing device analyzes the inputs. This can include using the sensors to triangulate the location of the VR floaters in three-dimensional space. This can also include processing the input using an algorithm to categorize the VR floaters, which are representative of real-life floaters, based on size, placement, movement, shape, etc. In some embodiments, the computing device also analyzes the patient's eye movements, as tracked by the sensors and cameras of the VR headset.

Additionally, the computing device compares the inputs about the VR floaters and the patient's eye movements to a database to evaluate the severity of the floaters and identify underlying causes of the floaters. The database constitutes inputs and eye movements from patients with known ocular conditions. In some embodiments, the computing device conducts this comparison in real-time as the patient reacts to the VR environment and creates the VR floaters.

The computing device might also generate a new VR environment or change the lighting condition of the existing VR environment. For example, the lighting might become brighter, the contrast might increase, the colors of the environment might become cooler, the blue in the environment might become more vivid, etc. In some embodiments, the characteristics of the changed lighting condition or the characteristics of the new VR environment may be determined by the algorithm based on the patient's eye movements or the input she provided. The new or adjusted VR environment can change how the floaters look to the patient, which provides the computing device and the physician with information about how intense the floaters are and what is causing the floaters to appear.

The VR headset will monitor the patient's eye movements as she reacts to the new or changed VR environment, and the patient will be prompted to recreate the VR floaters, this time based on the new environment or changed lighting conditions. The VR headset will collect these inputs, and the computing device will analyze the inputs and compare the inputs and the eye movements to the database.

In some embodiments, the VR method includes generating a report with details about the floaters and potential underlying causes. This report is generated by the computing device, and is, optionally, generated in real-time as the patient provides the input. The report can include images of the VR floaters that the patient created, data about how the patient's eyes moved in response to the different environments, data about the cause of the floaters, etc. In some embodiments, where the floaters appear to be related to ocular disorders, the report includes recommendations for further medical evaluations.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for detecting macular degeneration, the method comprising: administering a first visual test on screens of a virtual reality (VR) headset worn by a patient; collecting a first input regarding a response from the patient to the first visual test; administering a second visual test on the screen, wherein the second visual test comprises a different difficulty than the first visual test; collecting a second input regarding a response from the patient to the second visual test; and based on at least one of the first input or the second input, evaluating the patient for macular degeneration.

Clause 2. The method of Clause 1, further comprising collecting biodata and medical history from the patient.

Clause 3. The method of any of the preceding clauses, further comprising evaluating the biodata and the medical history to determine a difficulty of the first visual test and the different difficulty of the second visual test.

Clause 4. The method of any of the preceding clauses, wherein the first input and the second input comprise one or more of reaction times, eye movements, verbal statements, or pupil dilation.

Clause 5. The method of any of the preceding clauses, wherein collecting the first input and the second input comprises collecting the first input and the second input as the patient responds to the first visual test and the second visual test, respectively.

Clause 6. The method of any of the preceding clauses, further comprising evaluating the first input to determine the different difficulty of the second visual test.

Clause 7. The method of any of the preceding clauses, wherein the different difficulty of the second visual test is determined as the patient responds to the first visual test.

Clause 8. The method of any of the preceding clauses, wherein, if the patient provides correct responses to the first visual test, the second visual test is more difficult than the first visual test.

Clause 9. The method of any of the preceding clauses, wherein the different difficulty comprises one or more of a different contrast, brightness, or saturation.

Clause 10. The method of any of the preceding clauses wherein evaluating the patient for macular degeneration comprises comparing at least one of the first or second input with a database.

Clause 11. The method of any of the preceding clauses, wherein evaluating the patient for macular degeneration comprises comparing the first input with a database.

Clause 12. The method of any of the preceding clauses, wherein evaluating the patient for macular degeneration comprises comparing the second input with a database.

Clause 13. The method of any of the preceding clauses, further comprising predicting a likelihood of future macular degeneration, diagnosing the patient with macular degeneration, and recommending treatment.

Clause 14. A method for comparing macular degeneration in each eye of a patient, the method comprising: displaying a first image on a first screen of a virtual reality (VR) headset worn by the patient and a second image on a second screen of the VR headset, the second image being different from the first image; collecting a first input regarding the patient's perception of the first image and a second input regarding the patient's perception of the second image; implementing a first adjustment to the first image based on the first input and a second adjustment to the second image based on the second input; and calculating a first degree of macular degeneration in a first eye of the patient based on the first adjustment and a second degree of macular degeneration in a second eye based on the second adjustment, wherein the first eye perceives the first screen and the second eye of the patient perceives the second screen.

Clause 15. The method of Clause 14, further comprising collecting biodata and medical history from the patient.

Clause 16. The method of any of Clauses 14 to 15, wherein the first input and the second input comprise one or more of reaction times, eye movements, verbal statements, or pupil dilation.

Clause 17. The method of any of Clauses 14 to 16, wherein collecting the first input and the second input comprises collecting the first input and the second input as the patient perceives the first image and the second image, respectively.

Clause 18. The method of any of Clauses 14 to 17, wherein implementing the first adjustment and the second adjustment comprises changing one or more of a saturation, contrast, brightness, or acuity of the first image and the second image, respectively.

Clause 19. The method of any of Clauses 14 to 18, wherein calculating the first degree of macular degeneration in the first eye and the second degree of macular degeneration in the second eye comprises inversely correlating the first adjustment and the second adjustment with the first and second degrees of macular degeneration, respectively.

Clause 20. A system for detecting macular degeneration, the system comprising: a virtual reality (VR) headset having screens and at least one sensor configured to collect data about or input from a patient wearing the VR headset; and a computing device in electronic communication with the VR headset and being configured to process the data or the input and control an environment displayed on the screens based on the data or the input, wherein the computing device is configured to (a) cause a first visual test to be administered on the screens and receive a first input from the VR headset in response to the first visual test; (b) cause a second visual test to be administered on the screens and receive a second input from the VR headset in response to the second visual test; and (c) compare at least one of the first or second input with a database to evaluate the patient for macular degeneration.

Clause 21. The system of Clause 20, wherein the VR headset further comprises cameras and microphones configured to collect the data or the input from the patient.

Clause 22. The system of any of Clauses 20 to 21, wherein the data comprises biodata and medical history of the patient.

Clause 23. The system of any of Clauses 20 to 22, wherein the computing device further comprises a user interface at which a practitioner can view results as the computing device processes the data or the input to monitor the evaluation in real-time.

Clause 24. The system of any of Clauses 20 to 23, wherein the database comprises a compilation of known responses with known visual health statuses.

Clause 25. The system of any of Clauses 20 to 24, wherein the computing device is further configured to diagnose the patient with macular degeneration and recommend treatment.

Clause 26. A method for examining tear film quality, the method comprising: displaying visual stimuli on a screen of a virtual reality (VR) headset worn by a patient; collecting a sample of a tear film from an eye of the patient using a probe embedded in the VR headset; capturing images of the tear film before, during, and after display of the visual stimuli using cameras embedded in the VR headset; and comparing the sample and the images with a database to evaluate a patient's tear film.

Clause 27. The method of Clause 26, wherein displaying visual stimuli comprises inducing the tear film to form on the eye of the patient by guiding the patient to keep the eye open.

Clause 28. The method of any of Clauses 26 to 27, further comprising analyzing the sample using sensors embedded in the VR headset.

Clause 29. The method of any of Clauses 26 to 28, wherein analyzing sample comprises identifying an amount of salinity in the tear film.

Clause 30. The method of any of Clauses 26 to 29, wherein analyzing the sample comprises identifying a chemical composition of the tear film.

Clause 31. The method of any of Clauses 26 to 30, wherein analyzing the sample comprises analyzing the sample in real-time as the patient perceives the visual stimuli.

Clause 32. The method of any of Clauses 26 to 31, wherein capturing images comprises identifying an amount of particulate matter in the tear film.

Clause 33. The method of any of Clauses 26 to 32, wherein capturing images comprises identifying the tear film dynamics.

Clause 34. The method of any of Clauses 26 to 33, wherein capturing images comprises implementing an advanced image processing algorithm to process the images in real-time.

Clause 35. The method of any of Clauses 26 to 34, wherein comparing the sample and the images comprises comparing the sample and the images in real-time.

Clause 36. The method of any of Clauses 26 to 35, further comprising adjusting one or more of a brightness, a contrast, or a saturation of the visual stimuli if the patient has difficulty keeping the eye open.

Clause 37. A system for examining tear film quality, the system comprising: a virtual reality (VR) headset having screens, sensors, cameras, and probes; and a computing device in electronic communication with the VR headset, wherein the computing device is configured to: (a) cause visual stimuli to be displayed on the screens for a patient wearing the VR headset; (b) capture images of a patient's eyes before, while, and after the patient responds to the visual stimuli; and (c) compare the images with a database to evaluate a patient's tear film.

Clause 38. A virtual reality (VR) headset for examining tear film quality, the VR headset comprising: screens configured to display visual stimuli for a patient wearing the VR headset; a probe configured to collect a sample of a tear film from an eye of the patient; a plurality of sensors configured to analyze the sample; and a plurality of cameras configured to capture images of the tear film.

Clause 39. The VR headset of Clause 38, wherein the probe comprises a C-shaped hook, and a bend of the C-shaped hook contacts the eye of the patient to collect the sample of the tear film.

Clause 40. The VR headset of any of Clauses 38 to 39, wherein the probe is positioned within the VR headset such that, when the probe is not in use, the bend is within 0.5 mm of a front of an eyelid of the patient.

Clause 41. The VR headset of any of Clauses 38 to 40, wherein each camera of the plurality of cameras is positioned at an angle that is 45 degrees above a transverse plane of the eye.

Clause 42. The VR headset of any of Clauses 38 to 41, wherein each camera of the plurality of cameras is positioned at an angle that is between 30 degrees and 60 degrees above a transverse plane of the eye.

Clause 43. The VR headset of any of Clauses 38 to 42, wherein the plurality of cameras comprises two cameras directed at each eye.

Clause 44. The VR headset of any of Clauses 38 to 43, wherein each camera of the plurality of cameras is coupled with infrared illuminators.

Clause 45. The VR headset of any of Clauses 38 to 44, wherein each camera of the plurality of cameras has a diameter of less than 14.2 mm and produces at least 7× magnification.

Clause 46. A method for correcting a blink rate of a patient, the method comprising, by a virtual reality (VR) headset worn by the patient, conducting a visual task that induces eyes of the patient to move up and down.

Clause 47. A method for diagnosing and monitoring progression of retinal disorders, the method comprising: conducting a first scan of a retina of a patient using sensors and cameras embedded in a virtual reality (VR) headset worn by the patient; in response to conducting the first scan, isolating a first target area of the retina; administering a first vision test on screens of the VR headset; in response to administering the first vision test, detecting first retinal markers in the first target area with a probe embedded in the VR headset; while administering the first vision test, conducting a second scan of the retina; in response to conducting the second scan, isolating a second target area of the retina; administering a second vision test on the screens; in response to administering the second vision test, detecting second retinal markers in the second target area with the probe; and while administering the second vision test, conducting a third scan of the retina.

Clause 48. The method of Clause 47, wherein conducting the first, second, and third scans comprises conducting 360-degree scans of the retina that start and end at an optic nerve of the patient.

Clause 49. The method of any of Clauses 47 to 48, wherein conducting the first, second, and third scans comprises monitoring the one or more of eye movements, fixation stability, or response times of the patient.

Clause 50. The method of any of Clauses 47 to 49, wherein conducting the first and second scans comprises identifying the first and second target areas, the first and second target areas being areas of the retina that show signs of retinal disorders.

Clause 51. The method of any of Clauses 47 to 50, wherein conducting the first, second, and third scans comprises capturing ocular movements with sub-millimeter accuracy.

Clause 52. The method of any of Clauses 47 to 51, wherein administering the first and second vision tests comprises administering one or more of acuity level tests and color vision tests.

Clause 53. The method of any of Clauses 47 to 52, wherein administering the first and second vision tests comprises displaying both dynamic and static stimuli to stimulate different parts of the retina.

Clause 54. The method of any of Clauses 47 to 53, wherein administering the second vision test comprises transitioning from the first vision test before the patient completes the first vision test.

Clause 55. The method of any of Clauses 47 to 54, wherein the first and second vision tests stimulate the first and second target areas of the retina, respectively.

Clause 56. The method of any of Clauses 47 to 55, wherein detecting the first and second retinal markers comprises transmitting and receiving ultrasound signals from the probe.

Clause 57. The method of any of Clauses 47 to 56, wherein conducting the first, second, and third scans, isolating the first and second target areas, and detecting the first and second retinal markers occurs real-time as the patient responds to the first and second vision tests.

Clause 58. The method of any of Clauses 47 to 57, further comprising collecting a first input and a second input from the patient in response to the first and second vision tests, respectively.

Clause 59. The method of any of Clauses 47 to 58, further comprising comparing first and second input to a database.

Clause 60. The method of any of Clauses 47 to 59, further comprising comparing the first and second retinal markers to a database.

Clause 61. A virtual reality (VR) headset for diagnosing and monitoring progression of retinal disorders, the VR headset comprising: a plurality of sensors and a plurality of cameras, both the plurality of sensors and the plurality of cameras being configured to scan a retina of a patient wearing the VR headset; screens configured to display vision tests for the patient; and a probe configured to detect retinal markers on the retina.

Clause 62. The VR headset of Clause 61, wherein the plurality of sensors comprises a plurality of infrared sensors.

Clause 63. The VR headset of any of Clauses 61 to 62, wherein each infrared sensor of the plurality of infrared sensors has a pixel density of less than 8 μm.

Clause 64. The VR headset of any of Clauses 61 to 63, wherein each camera of the plurality of cameras is positioned at an angle that is 45 degrees above a transverse plane of an eye of the patient.

Clause 65. The VR headset of any of Clauses 61 to 64, wherein the probe comprises ultrasound sensors.

Clause 66. A system for diagnosing and monitoring progression of retinal disorders, the system comprising: a virtual reality (VR) headset having sensors, cameras, screens, and a probe; and a computing device in electronic communication with the VR headset, wherein the computing device is configured to (a) scan a retina of a patient wearing the VR headset, (b) isolate target areas of the retina, (c) administer vision tests on screens of the VR headset that stimulate the target areas, and (d) detect retinal markers in the target areas with the probe.

Clause 67. A method for assessing the rapid eye movement (REM) of a patient, the method comprising, by a virtual reality (VR) headset worn by the patient: while the patient is asleep, tracking eye movements of the patient with cameras and infrared sensors embedded in the VR headset; collecting eye movement data while the cameras and the infrared sensors track the eye movements; and processing the eye movement data to distinguish between different sleep stages to identify REM periods.

Clause 68. The method of Clause 67, wherein tracking the eye movements comprises continuously tracking the eye movements as the patient sleeps.

Clause 69. The method of any of Clauses 67 to 68, further comprising tracking breathing patterns of the patient with the cameras and the infrared sensors and collecting breathing pattern data while the cameras and the infrared sensors track the breathing patterns.

Clause 70. The method of any of Clauses 67 to 69, wherein processing the eye movement data comprises processing the data in real-time as the cameras and the infrared sensors track the eye movements.

Clause 71. The method of any of Clauses 67 to 70, further comprising generating a report that highlights irregularities in REM patterns of the patient.

Clause 72. The method of any of Clauses 67 to 71, wherein generating a report comprises recommending that the patient be evaluated for sleep-related disorders.

Clause 73. The method of any of Clauses 67 to 72, wherein generating a report comprises providing insights into overall eye health of the patient.

Clause 74. The method of any of Clauses 67 to 73, wherein generating a report comprises recommending changes a patient's sleep habits to increase a patient's quality of sleep.

Clause 75. The method of any of Clauses 67 to 74, wherein a latency between processing the eye movement data and generating report is less than 50 ms.

Clause 76. A system for administering sleep monitoring session to assess the rapid eye movement (REM) of a patient, the system comprising: a virtual reality (VR) headset having sensors and cameras, the sensors and cameras being configured to track eye movements of the patient while the patient is sleeping; and a computing device in electronic communication with the VR headset and being configured to process data collected while the sensors and the cameras track the eye movements, wherein the computing device is configured to (a) receive the data from the VR headset, and (b) process the data to distinguish between different sleep stages to identify REM periods.

Clause 77. The system of Clause 76, wherein the cameras are infrared cameras.

Clause 78. The system of any of Clauses 76 to 77, wherein the sensors and the cameras can track the eye movements with a margin of error of less than 2 mm.

Clause 79. The system of any of Clauses 76 to 78, wherein the sensors and the cameras can track the eye movements with a margin of error of less than 1.5 mm.

Clause 80. The system of any of Clauses 76 to 79, wherein the computing device comprises a user interface at which the patient can set up and initiate the sleep monitoring sessions.

Clause 81. The system of any of Clauses 76 to 80, wherein the user interface comprises portal for a physician to access the sleep monitoring session of the patient.

Clause 82. A virtual reality (VR) headset for assessing the rapid eye movement (REM) of a patient, the VR headset comprising a plurality of sensors and a plurality of cameras configured to track eye movements of the patient while the patient is sleeping.

Clause 83. The VR headset of Clause 82, wherein each camera of the plurality of cameras is positioned at an angle that is 45 degrees above a transverse plane of an eye of the patient.

### Clause 84. The VR headset of any of Clauses 82 to 83, wherein the cameras are infrared cameras.

Clause 85. The VR headset of any of Clauses 82 to 84, wherein the sensors and the cameras can track the eye movements with a margin of error of less than 2 mm.

Clause 86. The VR headset of any of Clauses 82 to 85, wherein the sensors and the cameras can track the eye movements with a margin of error of less than 1.5 mm.

Clause 87. A method for characterizing floaters perceived by the patient, the method comprising: generating a first lighting condition on screens of a virtual reality (VR) headset worn by a patient; monitoring eye movements of the patient using sensors and cameras embedded in the VR headset; prompting the patient to describe one or more virtual floaters; receiving input from the patient about the one or more virtual floaters; analyzing the input at a computing device in electronic communication with the VR headset; and comparing the eye movements and the input to a database.

Clause 88. The method of Clause 87, wherein prompting the patient to describe the one or more virtual floaters comprises prompting the patient to describe one or more of a size, shape, location, or movement of the floaters perceived by the patient.

Clause 89. The method of any of Clauses 87 to 88, wherein receiving the input about the one or more virtual floaters comprises receiving information from the patient about a size, density, and/or depth of the floaters perceived by the patient.

Clause 90. The method of any of Clauses 87 to 89, wherein prompting the patient to describe the one or more virtual floaters comprises prompting the patient to align the one or more virtual floaters with the floaters perceived by the patient.

Clause 91. The method of any of Clauses 87 to 90, wherein analyzing the input comprises using the sensors to triangulate the location of the floaters perceived by the patient in 3D space.

Clause 92. The method of any of Clauses 87 to 91, wherein analyzing the patient input comprises processing the input using an algorithm to categorize the floaters perceived by the patient based on characteristics and behavior.

Clause 93. The method of any of Clauses 87 to 92, wherein receiving the input and analyzing the input occurs in real-time as the patient describes the one or more virtual floaters.

Clause 94. The method of any of Clauses 87 to 93, further comprising analyzing the eye movements in real-time to identify potential underlying causes of the floaters perceived by the patient.

Clause 95. The method of any of Clauses 87 to 94, further comprising generating a second lighting condition, monitoring eye movements again, prompting the patient again, receiving a second input, and analyzing the second input.

Clause 96. The method of any of Clauses 87 to 95, wherein the second lighting condition comprises a different characteristic than the first lighting condition.

Clause 97. The method of any of Clauses 87 to 96, wherein the different characteristic comprises one or more of brightness, contrast, color temperature, or saturation.

Clause 98. The method of any of Clauses 87 to 97, wherein an algorithm determines the different characteristic based on the input and/or the second input.

Clause 99. The method of any of Clauses 87 to 98, further comprising generating a report detailing the types and severity of floaters and offering recommendations for further medical evaluation.

Clause 100. A virtual reality (VR) headset for characterizing floaters, the VR headset comprising: screens configured to display an environment with various lighting conditions for a patient wearing the VR headset; a plurality of sensors configured to monitor eye movements of the patient in response to the various lighting conditions and receive inputs from the patient, the inputs describing one or more floaters; and a plurality of cameras configured to monitor the eye movements.

Clause 101. The VR headset of Clause 100, wherein each camera of the plurality of cameras are infrared cameras.

Clause 102. The VR headset of any of Clauses 100 to 101, wherein each camera of the plurality of cameras is positioned at an angle that is 45 degrees above a transverse plane of an eye of the patient.

Clause 103. The VR headset of any of Clauses 100 to 102, wherein the VR headset further comprises microphones for receiving the inputs.

Clause 104. A system for characterizing floaters, the system comprising: a virtual reality (VR) headset having (a) screens configured to display an environment with various lighting conditions for a patient wearing the VR headset and (b) sensors and cameras configured to monitor eye movements of the patient and receive inputs from the patient, the inputs describing one or more floaters; and a computing device in electronic communication with the VR headset and being configured to analyze the eye movements and the inputs, wherein the computing device is configured to (a) receive data relating to the eye movements, (b) receive the inputs, and (c) compare the data and the inputs to a database to characterize the one or more floaters.

Clause 105. The system of Clause 104, further comprising a handheld device for receiving the inputs, the handheld device being in electronic communication with the VR headset and the computing device.

Clause 106. The system of any of Clauses 104 to 105, wherein the computing device comprises a user interface at which a physician can access the eye movements and inputs to monitor the process and adjust the lighting conditions.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise”as “comprise”is interpreted when employed as a transitional word in a claim.

As used herein, the term “about” is relative to the actual value stated, as will be appreciated by those of skill in the art, and allows for approximations, inaccuracies, and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,” such as “comprise”and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method for examining tear film quality, the method comprising:
displaying visual stimuli on a screen of a virtual reality (VR) headset worn by a patient;
collecting a sample of a tear film from an eye of the patient using a probe embedded in the VR headset;
capturing images of the tear film before, during, and after display of the visual stimuli using cameras embedded in the VR headset; and
comparing the sample and the images with a database to evaluate a patient's tear film.

2. The method of claim 1, wherein displaying visual stimuli comprises inducing the tear film to form on the eye of the patient by guiding the patient to keep the eye open.

3. The method of claim 1, further comprising analyzing the sample using sensors embedded in the VR headset.

4. The method of claim 3, wherein analyzing sample comprises identifying an amount of salinity in the tear film.

5. The method of claim 3, wherein analyzing the sample comprises identifying a chemical composition of the tear film.

6. The method of claim 3, wherein analyzing the sample comprises analyzing the sample in real-time as the patient perceives the visual stimuli.

7. The method of claim 1, wherein capturing images comprises identifying an amount of particulate matter in the tear film.

8. The method of claim 1, wherein capturing images comprises identifying the tear film dynamics.

9. The method of claim 1, wherein capturing images comprises implementing an advanced image processing algorithm to process the images in real-time.

10. The method of claim 1, wherein comparing the sample and the images comprises comparing the sample and the images in real-time.

11. The method of claim 1, further comprising adjusting one or more of a brightness, a contrast, or a saturation of the visual stimuli if the patient has difficulty keeping the eye open.

12. A virtual reality (VR) headset for examining tear film quality, the VR headset comprising:
screens configured to display visual stimuli for a patient wearing the VR headset;
a probe configured to collect a sample of a tear film from an eye of the patient;
a plurality of sensors configured to analyze the sample; and
a plurality of cameras configured to capture images of the tear film.

13. The VR headset of claim 12, wherein the probe comprises a C-shaped hook, and a bend of the C-shaped hook contacts the eye of the patient to collect the sample of the tear film.

14. The VR headset of claim 13, wherein the probe is positioned within the VR headset such that, when the probe is not in use, the bend is within 0.5 mm of a front of an eyelid of the patient.

15. The VR headset of claim 12, wherein each camera of the plurality of cameras is positioned at an angle that is 45 degrees above a transverse plane of the eye.

16. The VR headset of claim 12, wherein each camera of the plurality of cameras is positioned at an angle that is between 30 degrees and 60 degrees above a transverse plane of the eye.

17. The VR headset of claim 12, wherein the plurality of cameras comprises two cameras directed at each eye.

18. The VR headset of claim 12, wherein each camera of the plurality of cameras is coupled with infrared illuminators.

19. The VR headset of claim 12, wherein each camera of the plurality of cameras has a diameter of less than 14.2 mm and produces at least 7× magnification.

20. A method for correcting a blink rate of a patient, the method comprising, by a virtual reality (VR) headset worn by the patient, conducting a visual task that induces eyes of the patient to move up and down.
